(19) United States
(12) Patent Application Publication
(10) Pub. No.: US 2026/0253220 A1
(43) Pub. Date: Aug. 27, 2026

(54) SYSTEMS AND METHODS FOR WEAKLY SUPERVISED SEGMENTATION OF MEDICAL IMAGE DATA

(71) Applicant: GE Precision Healthcare LLC, Waukesha, WI (US)

(72) Inventors: István Megyeri, Szeged (HU); Valter Gila, Szeged (HU); András Marinovszki, Szeged (HU); László Ruskó, Budapest (HU); Lehel Mihály Ferenczi, Dunakeszi (HU)

(21) Appl. No.: 19/545,798

(22) Filed: Feb. 20, 2026

Related U.S. Application Data
(60) Provisional application No. 63/761,772, filed on Feb. 21, 2025.

(51) Int. Cl. G06T 7/00; G06T 7/11; G16H 30/40

(52) U.S. Cl. CPC G06T 7/0012; G06T 7/11; G16H 30/40; G06T 2207/20081

(57) ABSTRACT

In one embodiment, a system for weakly supervised segmentation of medical image data is provided. In various embodiments, the system can collect weak annotations for the medical image data; apply an interpolation technique to propagate the weak annotations to unannotated regions of the medical image data to generate propagated annotations; train a segmentation model using a training dataset comprising the weak annotations, the propagated annotations, and the medical image data; and iteratively retrain the segmentation model, wherein an iteration can comprise generating a set of pseudo labels for a subset of unannotated regions of the medical image data using the segmentation model; adding the set of pseudo labels to the training dataset; and updating the segmentation model using the training dataset, wherein pseudo labels generated from successive iterations are retained in the training dataset such that a number of annotations in the training dataset increases across successive iterations.

### CROSS REFERENCE TO RELATED APPLICATION

This application claims the benefit of and priority to U.S. Provisional Patent Application No. 63/761,772, filed on Feb. 21, 2025, the disclosure of which is incorporated herein by reference in its entirety.

### TECHNICAL FIELD

Techniques disclosed herein relate to medical image segmentation, and more particularly to weakly supervised segmentation of medical image data.

### BACKGROUND

Automatic segmentation of anatomical structures in medical image data has become increasingly reliant on deep learning models trained using annotated datasets. Although deep learning models have demonstrated substantial performance in segmenting medical image data, their effectiveness is often constrained by the heterogeneity in imaging protocols and sequences used during image acquisition. For instance, deep learning models trained using annotations derived from one magnetic resonance image (MRI) sequence, such as T1-weighted imaging (T1WI), frequently exhibit reduced segmentation accuracy when applied to other sequences, such as T2-weighted imaging (T2WI), even when targeting identical anatomical structures. Consequently, annotated datasets are created for each imaging protocol or sequence to achieve reliable segmentation accuracy, which can be time-consuming. Further, creating the annotated datasets involves extensive manual labeling by experts, resulting in increased cost, time consumption, and human effort.

Accordingly, systems or techniques that can efficiently generate training data for training a deep learning model for medical image segmentation can be considered as desirable.

### SUMMARY

This section is provided to introduce a selection of concepts that are further described below in the Detailed Description. This Brief Description is not intended to identify key or essential features of the claimed subject matter, nor is it intended to be used as an aid in limiting the scope of the claimed subject matter. Its sole purpose is to present concepts in a simplified form as a prelude to the more detailed description that is presented later. In one or more embodiments described herein, devices, systems, computer-implemented methods, apparatus or computer program products that facilitate weakly supervised segmentation of medical image data are described.

According to one or more embodiments, a system is provided. The system can comprise a non-transitory computer-readable memory that can store computer-executable components. The system can further comprise a processor that can be operably coupled to the non-transitory computer-readable memory and that can execute at least one of the computer executable components that can collect one or more weak annotations for the medical image data; apply an interpolation technique to propagate the one or more weak annotations to unannotated regions of the medical image data to generate propagated annotations; train a segmentation model using a training dataset comprising the one or more weak annotations, the propagated annotations, and the medical image data; and iteratively retrain the segmentation model. An iteration of retraining can comprise: generating a set of pseudo labels for a subset of unannotated regions of the medical image data using the segmentation model; adding the set of pseudo labels to the training dataset; and updating one or more parameters of the segmentation model using the training dataset, wherein the set of pseudo labels generated from successive iterations can be retained in the training dataset such that a number of annotations associated with the medical image data in the training dataset increases across successive iterations.

According to one or more embodiments, a computer-implemented method is provided. The computer-implemented method can comprise collecting, by a system operatively coupled to a processor, one or more weak annotations for medical image data; applying, by the system, an interpolation technique to propagate the one or more weak annotations to unannotated regions of the medical image data to generate propagated annotations; training, by the system, a segmentation model using a training dataset comprising the one or more weak annotations, the propagated annotations, and the medical image data; and iteratively retraining the segmentation model. An iteration of retraining can comprise: generating a set of pseudo labels for a subset of unannotated regions of the medical image data using the segmentation model; adding the set of pseudo labels to the training dataset; and updating one or more parameters of the segmentation model using the training dataset, wherein the set of pseudo labels generated from successive iterations can be retained in the training dataset such that a number of annotations associated with the medical image data in the training dataset increases across successive iterations.

According to one or more embodiments, a computer program product for weakly supervised segmentation of medical image data is provided. The computer program product can comprise a non-transitory computer-readable memory having program instructions embodied therewith, the program instructions executable by a processor to cause the processor to collect, by the processor, one or more weak annotations for the medical image data; apply, by the processor, an interpolation technique to propagate the one or more weak annotations to unannotated regions of the medical image data to generate propagated annotations; train, by the processor, a segmentation model using a training dataset comprising the one or more weak annotations, the propagated annotations; and iteratively retrain, by the processor, the segmentation model. An iteration of retraining can comprise: generating a set of pseudo labels for a subset of unannotated regions of the medical image data using the segmentation model; adding the set of pseudo labels to the training dataset; and updating one or more parameters of the segmentation model using the training dataset, wherein the set of pseudo labels generated from successive iterations can be retained in the training dataset such that a number of annotations associated with the medical image data in the training dataset increases across successive iterations.

Various other features, objects, and advantages of the invention will be made apparent from the following description taken together with the drawings.

### BRIEF DESCRIPTION OF THE DRAWINGS

The present disclosure will be better understood from reading the following description of non-limiting examples, with reference to the attached drawings broadly described below:

FIG. 1 illustrates a block diagram of an example, non-limiting system that can facilitate weakly supervised segmentation of medical image data in accordance with one or more embodiments described herein.

FIG. 2 illustrates example, non-limiting medical image data with weak annotations in accordance with one or more embodiments described herein.

FIG. 3 illustrates example, non-limiting medical image data with weak annotations in accordance with one or more embodiments described herein.

FIG. 4 illustrates example, non-limiting medical image data showing how weak annotations can be propagated to unannotated regions of the medical image data in accordance with one or more embodiments described herein.

FIG. 5 illustrates an example, non-limiting medical image showing how weak annotations can be propagated to unannotated regions of the medical image in accordance with one or more embodiments described herein.

FIG. 6 illustrates an example, non-limiting block diagram of a training dataset comprising weak annotations for training a segmentation model in accordance with one or more embodiments described herein.

FIG. 7 illustrates an example, non-limiting block diagram showing how a segmentation model can be trained on weak annotations in accordance with one or more embodiments described herein.

FIG. 8 illustrates an example, non-limiting block diagram of a training dataset comprising weak annotations and pseudo labels for training a segmentation model in accordance with one or more embodiments described herein.

FIG. 9 illustrates a flow diagram of an example, non-limiting computer-implemented method that can facilitate weakly supervised segmentation of medical image data in accordance with one or more embodiments described herein.

FIG. 10 illustrates a flow diagram of an example, non-limiting computer-implemented method that can facilitate weakly supervised segmentation of medical image data in accordance with one or more embodiments described herein.

FIG. 11 illustrates charts showing model performance of a segmentation model trained on weak annotations in accordance with one or more embodiments described herein.

FIG. 12 illustrates a block diagram of an example, non-limiting computing device that can facilitate segmentation of medical image data using weak annotations in accordance with one or more embodiments described herein.

FIG. 13 illustrates a block diagram of an example, non-limiting operating environment in which one or more embodiments described herein can be facilitated.

FIG. 14 illustrates an example networking environment operable to execute various implementations described herein.

The drawings illustrate specific aspects of the described components, systems and methods for segmenting medical image data. Together with the following description, the drawings demonstrate and explain the principles of the structures, methods, and principles described herein. In the drawings, the thickness and size of components may be exaggerated or otherwise modified for clarity. Well-known structures, materials, or operations are not shown or described in detail to avoid obscuring aspects of the described components, systems and methods.

### DETAILED DESCRIPTION

The following detailed description is merely illustrative and is not intended to limit embodiments or application/uses of embodiments. Furthermore, there is no intention to be bound by any expressed or implied information presented in the preceding Background or Summary sections, or in the Detailed Description section.

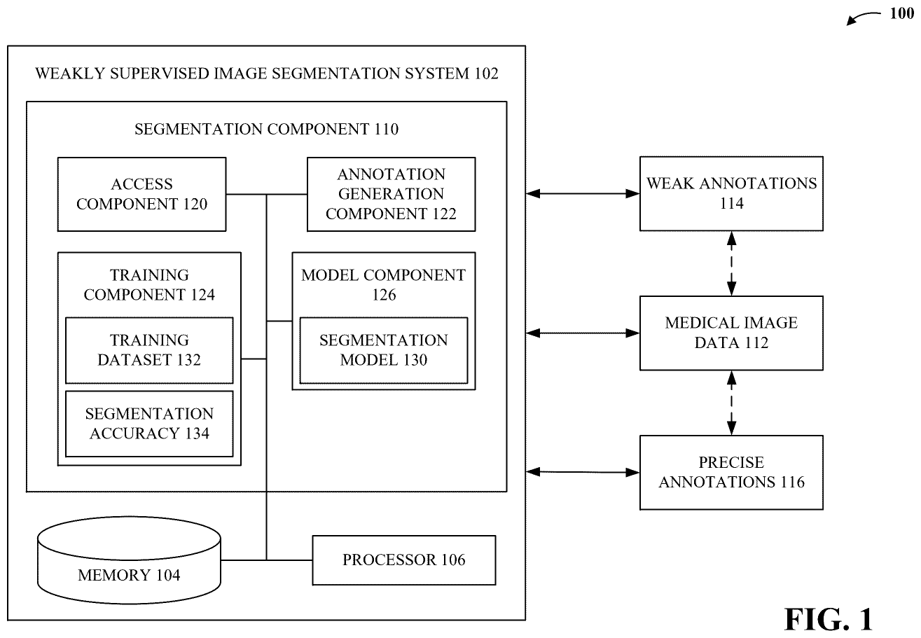

Embodiments of the present disclosure will now be described, by way of example, with reference to FIGS. 1-14, in which the following description relates to various examples of a system and method for training segmentation models on weak annotations.

### DEFINITIONS

Precise annotations: Precise annotations are labeling that accurately delineates boundaries and/or locations of target anatomical structures at a pixel or voxel level, with minimal inclusion of non-target tissue and high anatomical consistency.

Weak annotations: Weak annotations are labeling that provides limited, coarse, or incomplete information about target anatomical structures, such as image-level labels, bounding boxes, sparse points, rough contours, or partial markings, rather than precise pixel-or voxel-level delineations. As used herein, the weak annotations can include any annotations that are quicker to create than precise annotations (e.g., quicker than full mask preparation).

Imaging protocol: An imaging protocol refers to a defined set of acquisition parameters and procedural settings used during image acquisition for a given modality, including factors such as contrast administration, timing, resolution, and scanner-specific settings.

Imaging sequence: An imaging sequence refers to a specific data acquisition scheme within a given modality, particularly MRI, that determines image contrast characteristics and tissue appearance, such as T1-weighted or T2-weighted sequences.

For given medical image data (e.g., one or more computed tomography (CT) scanned images, one or more magnetic resonance imaging (MRI) scanned images, one or more X-ray scanned images, one or more positron emission tomography (PET) scanned images, one or more ultrasound scanned images, or a combination thereof), it can be desired to segment the medical image data. For instance, it can be desirable to generate one or more segmentation masks that delineate one or more anatomical structures, tissue types, lesions, or other regions of interest in the medical image data (e.g., identify boundaries between different tissues, isolate pathological features, highlight clinically relevant areas).

Unfortunately, various existing techniques to segment medical image data via deep learning consume excessive time and amounts of manual intervention to create training datasets. Specifically, traditional deep learning-based segmentation models often demand extensive annotated datasets, where such annotated datasets must be manually labeled by experts to identify anatomical structures or regions of interest. This consumes significant time and human effort to construct sufficiently large training datasets for training the deep learning-based segmentation models. Further, to sufficiently train deep learning-based segmentation models, the annotated datasets typically contain precise and complete annotations of target anatomical structures, thereby further increasing the manual effort, cost, and expertise required to generate the training datasets.

Additionally, conventional deep learning-based segmentation models are trained using protocol-specific datasets (e.g., contrast-enhanced versus non-contrast imaging, acquisition parameter variations) or sequence-specific datasets (e.g., T1-weighted imaging (T1WI), T2-weighted imaging (T2WI), fluid-attenuated inversion recovery (FLAIR)). That is, deep learning models are often limited to segmenting images of a particular imaging protocol or imaging sequence, constraining the ability of the deep learning model to perform accurately on medical image data acquired using different imaging protocols or imaging sequences. In other words, such conventional deep learning-based segmentation models lack adaptability across different imaging protocols or imaging sequences. For example, deep learning models trained on T1WI suffers a significant drop in performance when applied on T2WI of the same structure or vice-versa

Although some deep learning-based segmentation techniques utilize models trained across multiple imaging protocols or sequences, such techniques often necessitate large and diverse annotated training datasets or extensive retraining and tuning to accommodate variability across protocols or sequences. As stated previously, creating such large and diverse annotated training datasets consumes significant amounts of time and human effort.

Accordingly, systems or techniques that can efficiently generate training data for training a segmentation model to segment medical image data across different protocols or sequences without involving extensive manual intervention for data annotation can be considered as desirable.

Various embodiments described herein can address one or more of these technical problems. One or more embodiments described herein include systems, computer-implemented methods, apparatus, or computer program products that can facilitate weakly supervised segmentation of medical image data. More specifically, the various embodiments described herein involve propagation of weak annotations to unannotated regions of the medical image data, iterative generation of annotations via the segmentation model based on weak annotations, and iterative training of the segmentation model on pseudo labels for segmenting medical image data, which does not involve a time-consuming and manual-intervention-based data annotation and does not consume excessive amounts of computer processing capacity for training a segmentation model for each imaging protocol or sequence. More specifically still, the various embodiments herein can reduce the amount of computing resources and manual intervention demanded by existing techniques.

Various embodiments described herein can be considered as a computerized tool (e.g., any suitable combination of computer-executable hardware or computer-executable software) that can facilitate weakly supervised segmentation of medical image data. In various aspects, such computerized tool can comprise an access component, a data generation component, a training component, and/or a model component.

In various embodiments, there can be medical image data. In various aspects, the medical image data can depict one or more anatomical structures (e.g., tissues, organs, body parts, or portions thereof) of a medical patient (e.g., human, animal, or otherwise). In various instances, the medical image data can comprise one or more medical images that exhibit any suitable size, format, or dimensionality (e.g., can be a two-dimensional pixel array, can be a three-dimensional voxel array). In various cases, the medical image data can be generated or otherwise captured by any suitable medical imaging modality (e.g., by a CT scanner, by an MRI scanner, by an X-ray scanner, by a PET scanner, or by an ultrasound scanner). In various aspects, the medical image data can have undergone any suitable image reconstruction technique (e.g., filtered back projection).

In any case, it can be desired to perform any suitable segmentation on the medical image. Non-limiting examples of such segmentation can include organ segmentation (e.g., identifying and delineating specific organs within a medical image), tissue-type segmentation (e.g., distinguishing between different tissue classes such as muscle, fat, or bone), lesion or pathology segmentation (e.g., isolating tumors, cysts, or other abnormal regions), or multi-class segmentation (e.g., simultaneously segmenting multiple anatomical structures or regions of interest). In various aspects, the computerized tool described herein can facilitate such segmentation with respect to the medical image data.

In various embodiments, the access component of the computerized tool can electronically receive or otherwise electronically access the medical image data. Likewise, the access component can electronically receive or otherwise electronically access one or more weak annotations for the medical image data. In some aspects, the access component can electronically retrieve the medical image data and the one or more weak annotations from any suitable centralized or decentralized data structures (e.g., graph data structures, relational data structures, hybrid data structures), whether remote from or local to the access component. For example, the access component can retrieve the medical image data from whatever medical imaging device generated or captured the medical image. In any case, the access component can electronically obtain or access the medical image data and the one or more weak annotations, such that other components of the computerized tool can electronically interact with (e.g., read, write, edit, copy, manipulate) the medical image data and the one or more weak annotations.

In some instances, the access component can electronically receive or otherwise electronically access one or more precise annotations for the medical image data. such that other components of the computerized tool can electronically interact with (e.g., read, write, edit, copy, manipulate) the one or more precise annotations. In various aspects, the medical image data, the one or more weak annotations, and, in some cases, the one or more precise annotations can make up a training dataset for training a segmentation model (e.g., a deep learning neural network for segmenting medical image data).

In any case, the data generation component of the computerized tool can propagate the one or more weak annotations to unannotated regions of the medical image data. Specifically, the data generation component can apply any suitable interpolation technique to the one or more weak annotations to generate additional weak annotations for the medical image data. In instances where there are one or more precise annotations for the medical image data, the data generation component can apply any suitable interpolation technique to the one or more weak annotations and the one or more precise annotations to generate additional annotations (e.g., propagated annotations) for the medical image data.

In various instances, the training component of the computerized tool can train the segmentation model on the medical image data and the one or more weak annotations (e.g., and, in some cases, the one or more precise annotations) to generate a segmented image for inputted medical image data. In particular, the training component can feed the medical image data and the one or more weak annotations (e.g., and, in some cases, the one or more precise annotations) to a segmentation model, which can cause the segmentation model to produce a set of pseudo labels for the medical image data. Then, the training component can train the segmentation model on the medical image data, the one or more weak annotations, and the set of pseudo labels. That is, the training component can iteratively generate pseudo labels that can be included in the training dataset, and the training component can iteratively train the segmentation model on the one or more weak annotations and the set of pseudo labels. In this manner, the training component can, in each training iteration, generate new annotations for the medical image data that can be used to train the segmentation model until a desirable segmentation accuracy is achieved. Thereafter, in various embodiments, the model component of the computerized tool can execute the segmentation model on inputted medical image data that can be desirable to segment.

This training approach can consume significantly less time to construct the training dataset for reliably training the segmentation model than existing training methods that rely on precise annotations. That is, such weakly supervised training can achieve performance comparable to training on precise annotations while significantly reducing the manual annotation time required.

Various embodiments described herein can be employed to use hardware or software to solve problems that are highly technical in nature (e.g., to facilitate weakly supervised image segmentation), that are not abstract and that cannot be performed as a set of mental acts by a human. Further, some of the processes performed can be performed by a specialized computer (e.g., a segmentation model comprising a deep learning neural network architecture) for carrying out defined acts related to weakly supervised image segmentation. For example, such defined acts can include: accessing, by a device operatively coupled to a processor, medical image data and corresponding weak annotations; applying an interpolation technique to propagate the corresponding weak annotations to regions of the medical image data without annotations; generating, by the device and via execution of a segmentation model on the medical image data and corresponding weak annotations, a set of pseudo labels for the medical image data; iteratively generating, by the device and via execution of the segmentation model on the medical image data, the corresponding weak annotations, and the set of pseudo labels for the medical image data, additional pseudo labels; generating, by the device and via execution of the segmentation model on inputted medical image data, a segmented image of the inputted medical image data; and rendering, by the device, the segmented image of the medical image on an electronic display.

Such defined acts are not performed manually by humans. Indeed, neither the human mind nor a human with pen and paper can: electronically access a medical image (e.g., a CT scanned image, an MRI scanned image, an X-ray scanned image) and corresponding weak annotations; electronically apply an interpolation technique to propagate the corresponding weak annotations to regions of the medical image data without annotations; electronically execute a segmentation model on the medical image data, corresponding weak annotations, and pseudo labels for the medical image data; electronically generate a segmented image based on the execution of the segmentation model on inputted medical image data; and electronically display such segmented image of the inputted medical image data on a computer screen. Indeed, a segmentation model is an inherently-computerized construct that simply cannot be implemented in any way by the human mind without computers. Similarly, medical image data is an inherently computerized construct that are generated or captured by electronic medical hardware (e.g., CT scanners, MRI scanners, X-ray scanners, PET scanners, ultrasound scanners) and not in any way by the human mind without computers. Accordingly, a computerized tool that can execute a segmentation model on medical image data and corresponding weak annotations to generate pseudo labels or segmented images is likewise inherently-computerized and cannot be implemented in any sensible, practical, or reasonable way without computers.

Moreover, various embodiments described herein can integrate into a practical application various teachings relating to weakly supervised medical image segmentation. As explained above, it can be desired to segment medical image data (e.g., to delineate regions of interest in medical images). Some existing techniques to perform such segmentation consume excessive processing time and require extensive amounts of manual annotation effort. For instance, such existing techniques involve extensive annotated datasets comprising precise annotations for training a segmentation model, where such annotated datasets must be manually labeled by experts to identify anatomical structures or regions of interest, thereby consuming significant time and human effort. Other existing techniques further struggle to generalize across diverse imaging protocols or sequences. Such techniques are often trained on protocol-specific or sequence-specific datasets (e.g., T1W1 or T2W2), which limit their ability to perform accurately on medical images acquired using different imaging protocols or sequences. Moreover, the segmentation performed in such existing techniques typically operates in isolation, relying on protocol or sequence-specific parameters or architectures, and therefore lack adaptability or interoperability across heterogeneous datasets without training on extensive annotated data.

Various embodiments described herein can address one or more of these technical problems. Specifically, the various embodiments described herein can collect one or more weak annotations of medical image data for training a segmentation model. That is, the one or more weak annotations and the medical image data can form a training dataset for the segmentation model. In various cases, the embodiments herein can propagate the one or more weak annotations to unannotated regions of the medical image data by applying an interpolation technique. By propagating the one or more weak annotations to unannotated regions, the embodiments herein can expand the training dataset without incurring additional manual annotation effort. In any case, the segmentation model can be initially training on the training dataset generated from the one or more weak annotations and the medical image date. Following the initial training, the segmentation model can be executed on the medical image data to generate pseudo labels associated with the medical image data. The pseudo labels can then be used to train the segmentation model. That is, the segmentation model can be further trained on the medical image data and the pseudo labels. In other words, the pseudo labels can be added to the training dataset for training the segmentation model. By adding the pseudo labels to the training dataset and further training the segmentation model on such training dataset, embodiments herein can gradually expand the training dataset (e.g., increase the number of annotations associated with the medical image data). Specifically, embodiments herein can iteratively generate the pseudo labels by iteratively executing the segmentation model on the one or more weak annotations and previously generated pseudo labels (e.g., pseudo labels from a previous training iteration). In other words, the segmentation model can be iteratively trained on the pseudo labels, wherein additional pseudo labels are generated in each training iteration. By training the segmentation model in this fashion, the training dataset can gradually expand in each training iteration by adding the resulting generated pseudo labels. In other words, the embodiments herein can iteratively and gradually generate labels or annotations associated with the medical image data, which can be used to further tune or retrain the segmentation model. Thus, the segmentation model can be iteratively retrained and tuned to achieve a desired segmentation accuracy while being trained on weak annotations instead of precise annotations, thereby significantly reducing the annotation time to achieve a reliable segmentation accuracy. Such embodiments certainly constitute concrete and tangible technical improvements in the field of image processing, and thus such embodiments clearly qualify as useful and practical applications of computers.

Furthermore, various embodiments described herein can control real-world tangible devices based on the disclosed teachings. For example, various embodiments described herein can electronically execute real-world deep learning neural networks (e.g., segmentation models) on real-world medical images (e.g., CT images, MRI images, X-ray images, PET images, ultrasound images), and can electronically render any results produced by such real-world deep learning neural networks (e.g., segmented images) on real-world computer screens.

It should be appreciated that the herein figures and description provide non-limiting examples of various embodiments and are not necessarily drawn to scale.

FIG. 1 illustrates a block diagram of an example, non-limiting system 100 that can facilitate weakly supervised segmentation of medical image data in accordance with one or more embodiments described herein.

In various embodiments, a weakly supervised image segmentation system 102 can comprise a processor 106 (e.g., computer processing unit, microprocessor) and a non-transitory computer-readable memory 104 that is operably or operatively or communicatively connected or coupled to the processor 106. The non-transitory computer-readable memory 104 can store computer-executable instructions which, upon execution by the processor 106, can cause the processor 106 or other components of the weakly supervised image segmentation system 102 (e.g., segmentation component 110, access component 120, annotation generation component 122, training component 124, and/or model component 126) to perform one or more acts. In various embodiments, the non-transitory computer-readable memory 104 can store computer-executable components (e.g., segmentation component 110, access component 120, annotation generation component 122, training component 124, and/or model component 126), and the processor 106 can execute the computer-executable components. In various aspects, segmentation component 110 can comprise access component 120, annotation generation component 122, training component 124, and/or model component 126.

In various embodiments, it can be desired to perform any suitable image segmentation on inputted medical image data. As a non-limiting example, the image segmentation can comprise anatomical segmentation, which can involve delineating or labeling anatomical structures such as organs, tissues, or bones depicted in the inputted medical image data. As another non-limiting example, the image segmentation can comprise pathological segmentation, which can involve identifying and outlining regions associated with abnormalities, such as lesions, tumors, or other pathological features. As yet another non-limiting example, the image segmentation can comprise functional or physiological segmentation, which can involve distinguishing regions based on physiological activity or function as represented in the inputted medical image data. As still another non-limiting example, the image segmentation can comprise vessel or boundary segmentation, which can involve isolating vascular structures or structural boundaries within the inputted medical image data. Further, it can be desired to efficiently train a segmentation model to perform such medical image segmentation.

Accordingly, the weakly supervised image segmentation system 102 can efficiently train a segmentation model to perform such medical image segmentation on inputted medical image data via weakly supervised training as described herein.

In various embodiments, the weekly supervised image segmentation system 102 can comprise an access component 120. In various aspects, the access component 120 can electronically receive or otherwise electronically access medical image data 112 and weak annotations 114 for the medical image data 112. In various instances, the access component 120 can electronically retrieve medical image data 112 and weak annotations 114 from any suitable centralized or decentralized data structures (not shown) or from any suitable centralized or decentralized computing devices (not shown). As a non-limiting example, whatever medical imaging device, equipment, or modality (e.g., CT scanner, MRI scanner, X-ray scanner, PET scanner, ultrasound scanner) that generated or captured the medical image data 112 can transmit the medical image data 112 to the access component 120. As another non-limiting example, any annotation generation system, annotation platform, or annotation interface (e.g., clinician annotation workstation, automated annotation algorithm, semi-automated labeling tool, or crowdsourcing annotation system) that generated or produced the weak annotations 114 can transmit the weak annotations 114 to the access component 120. In any case, the access component 120 can electronically obtain or access the medical image data 112 and weak annotations 114, such that other components of the weakly supervised image segmentation system 102 can electronically interact with the medical image data 112 and weak annotations 114.

In some instances, the access component 120 can electronically receive or otherwise electronically access one or more precise annotations 116 for the medical image data 112. In various instances, the access component 120 can electronically retrieve the one or more precise annotations 116 from any suitable centralized or decentralized data structures (not shown) or from any suitable centralized or decentralized computing devices (not shown). As a non-limiting example, any annotation generation system, annotation platform, or annotation interface (e.g., clinician annotation workstation, automated annotation algorithm, semi-automated labeling tool, or crowdsourcing annotation system) that generated or produced the one or more precise annotations 116 can transmit the one or more precise annotations 116 to the access component 120. In any case, the access component 120 can electronically obtain or access the one or more precise annotations 116, such that other components of the weakly supervised image segmentation system 102 can electronically interact with the one or more precise annotations 116.

In various embodiments, the medical image data 112 can comprise one or more medical images that can depict any suitable anatomical structure of any suitable medical patient. As some non-limiting examples, the anatomical structure can be any suitable tissue of the medical patient (e.g., bone tissue, lung tissue, muscle tissue, brain tissue), any suitable organ of the medical patient (e.g., heart, liver, lung, brain, eye, colon, blood vessel), any suitable bodily fluid of the medical patient (e.g., blood, amniotic fluid), any other suitable body part of the medical patient, or any suitable portion thereof.

In various aspects, the medical image data 112 can exhibit any suitable format, size, or dimensionality. As a non-limiting example, the medical image data 112 can comprise one or more medical images that are two-dimensional array of pixels. As another non-limiting example, the medical image data 112 can comprise one or more medical images that are three-dimensional array of voxels.

In various instances, the medical image data 112 can be generated or otherwise captured by any suitable medical imaging device, medical imaging equipment, or medical imaging modality (not shown). As a non-limiting example, the medical image data 112 can be generated or otherwise captured by a CT scanner, in which case the medical image data 112 can be considered as a CT scanned image. As another non-limiting example, the medical image data 112 can be generated or otherwise captured by an MRI scanner, in which case the medical image data 112 can be considered as an MRI scanned image. As yet another non-limiting example, the medical image data 112 can be generated or otherwise captured by a PET scanner, in which case the medical image data 112 can be considered as a PET scanned image. As still another non-limiting example, the medical image data 112 can be generated or otherwise captured by an X-ray scanner, in which case the medical image data 112 can be considered as an X-ray scanned image. As even another non-limiting example, the medical image data 112 can be generated or otherwise captured by an ultrasound scanner, in which case the medical image data 112 can be considered as an ultrasound scanned image. Moreover, the medical image data 112 can have undergone any suitable image reconstruction techniques, such as filtered back projection.

In various aspects, the medical image data 112 can be generated or otherwise captured according to any suitable imaging protocol. As a non-limiting example, when the medical image data 112 is generated or otherwise captured by a CT scanner, the medical image data 112 can be generated or otherwise captured according to a helical acquisition protocol, in which case the medical image data 112 can be considered as helical CT image data. As another non-limiting example, the medical image data 112 can be generated according to a low-dose acquisition protocol, in which case the medical image data 112 can be considered as low-dose CT image data. As yet another non-limiting example, the medical image data 112 can be generated according to a contrast-enhanced acquisition protocol, in which case the medical image data 112 can be considered as contrast-enhanced medical image data.

In various cases, the medical image data 112 can be generated or otherwise captured according to any suitable imaging sequence (or pulse sequence). As a non-limiting example, when the medical image data 112 is generated or otherwise captured by an MRI scanner, the medical image data 112 can be generated according to a T1-weighted pulse sequence, in which case the medical image data 112 can be considered as T1-weighted MRI image data. As another non-limiting example, the medical image data 112 can be generated according to a T2-weighted pulse sequence, in which case the medical image data 112 can be considered as T2-weighted MRI image data. As yet another non-limiting example, the medical image data 112 can be generated according to a diffusion-weighted imaging sequence, in which case the medical image data 112 can be considered as diffusion-weighted MRI image data.

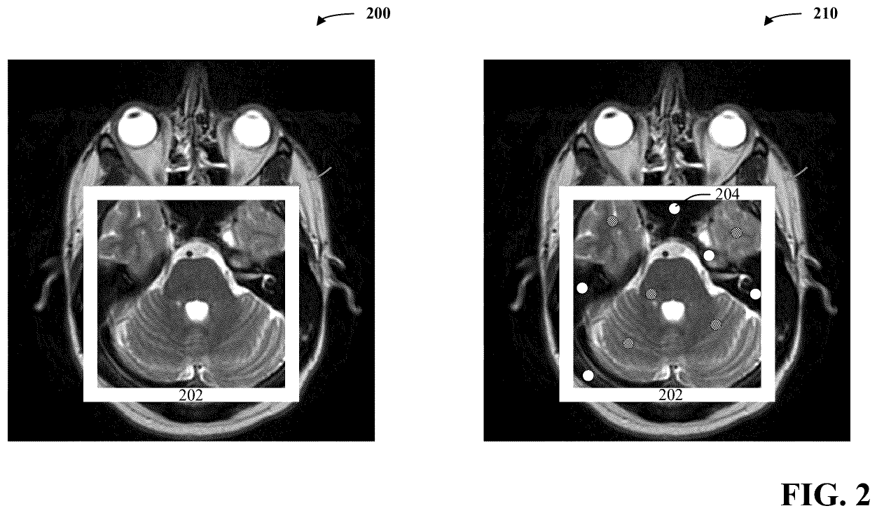

In various embodiments, the weak annotations 114 can correspond to the medical image data 112. In various aspects, the weak annotations 114 can indicate approximate locations, classifications, or descriptive labels associated with one or more regions depicted within the medical image data 112. Weak annotations 114 can comprise any suitable annotation types. As a non-limiting examples, the weak annotations 114 can comprise, but are not limited to, bounding boxes (two-dimensional or three-dimensional), point-based annotations with an inner or outer label, one or more slices that include complete segmentation, scribble-based annotations, region-level labels, image-level classification labels, presence indicators associated with one or more anatomical structures, or combinations thereof. In various cases, the data generation component 124 can assign a background label to negative slices of the medical image data 112. Specifically, data generation component 124 can assign a background label to slices in the medical image data 112 to indicate that the slice does not contain the target structure or feature of interest. In some instances, the weak annotations 114 can represent coarse, partial, or non-pixel-level information that identifies whether a target feature is present within the medical image data 112. In other instances, the weak annotations 114 can include textual metadata, categorical identifiers, or structured clinical descriptors that are associated with the medical image data 112. Further, weak annotations 114 can be incomplete annotations. That is, weak annotations 114 can omit one or more portions of an anatomical structure, pathological region, or feature depicted within the medical image data 112, and can instead identify only a subset of the anatomical structure, pathological region, or feature. In some instances, the weak annotations 114 can provide partial spatial information, partial labeling information, or selectively labeled slices or regions, while other portions of the medical image data 112 remain unlabeled or unannotated. Non-limiting examples of medical image data 112 and weak annotations 114 are described with respect to FIGS. 2-4.

In various embodiments, the weekly supervised image segmentation system 102 can comprise a training component 124. In various aspects, training component 124 can construct, as described herein, a training dataset 132 for training a segmentation model 130. Specifically, training component 124 can construct training dataset 132 from medical image data 112 and weak annotations 114. In other words, training dataset 132 can comprise medical image data 112 and weak annotations 114. In some instances, the training dataset 132 can further comprise the one or more precise annotations 116 if available.

In various embodiments, the weakly supervised image segmentation system 102 can comprise a annotation generation component 122. In various aspects, annotation generation component 122 can, as described herein, apply an interpolation technique to propagate weak annotations 114 to unannotated regions of medical image data 112. Annotation generation component 122 can apply any suitable interpolation technique to propagate the weak annotations 114. As a non-limiting example, annotation generation component 122 can apply nearest-neighbor interpolation. Specifically, annotation generation component 122 can, for one or more unannotated regions in the medical image data 112, identify a nearest labeled region (e.g., a nearest weak annotation from weak annotations 114), and assign the unannotated region the same label as the nearest labeled region. As a non-limiting example, annotation generation component 122 can apply a support vector machine (SVM) technique. For instance, annotation generation component 122 can use the weak annotations 114 to train an SVM on feature values to determine separating boundaries between different label classes and apply the SVM on one or more unannotated regions in the medical image data 112 to predict corresponding labels for the one or more unannotated regions. In various instances, the interpolation technique can also be applied to propagate the one or more precise annotations 116 to unannotated regions of medical image data 112, if the one or more precise annotations 116 are available.

In any case, annotation generation component 122 can generate additional annotations (e.g., propagated annotations) for medical image data 112 via the interpolation technique. The interpolation technique can be performed without additional manual annotation effort. That is, annotation generation component 122 can propagate weak annotations 114, and in some cases the one or more precise annotations 116, to unannotated regions of medical image data 112 to generate propagated annotations without increasing annotation time. In various aspects, training component 124 can add the additional propagated annotations generated from applying the interpolation technique to the training dataset 132. As used herein, weak annotations 114 can include the additional propagated annotations generated by applying the interpolation technique. That is, as used herein, weak annotations 114 can refer to the initially collected weak annotations and the propagated annotations (e.g., the annotations propagated to unannotated regions of medical image data 112.) Non-limiting examples of propagating weak annotations 114 to unannotated regions of medical image data 112 are described with respect to FIGS. 2-5.

In various embodiments, the weekly supervised image segmentation system 102 can comprise a model component 126. In various aspects, the model component 126 can electronically store, electronically maintain, electronically control, or otherwise electronically access segmentation model 130. In various aspects, the segmentation model 130 can have or otherwise exhibit any suitable internal architecture. For example, the segmentation model can be a deep learning neural network including an input layer, one or more hidden layers, and an output layer. In various instances, any of such layers can be coupled together by any suitable interneuron connections or interlayer connections, such as forward connections, skip connections, or recurrent connections. Furthermore, in various cases, any of such layers can be any suitable types of neural network layers having any suitable learnable or trainable internal parameters. For example, any of such input layer, one or more hidden layers, or output layer can be convolutional layers, whose learnable or trainable internal parameters can be convolutional kernels. As another example, any of such input layer, one or more hidden layers, or output layer can be dense layers, whose learnable or trainable internal parameters can be weight matrices or bias values. As still another example, any of such input layer, one or more hidden layers, or output layer can be batch normalization layers, whose learnable or trainable internal parameters can be shift factors or scale factors. Further still, in various cases, any of such layers can be any suitable types of neural network layers having any suitable fixed or non-trainable internal parameters. For example, any of such input layer, one or more hidden layers, or output layer can be non-linearity layers, padding layers, pooling layers, or concatenation layers.

In various cases, the segmentation model 130 can be a two-dimensional or a three-dimensional segmentation model, depending on the dimensionality of the medical image data 112.

In any case, training component 124 can, as described herein, train the segmentation model 130 on training dataset 132. That is, training component 124 can train the segmentation model 130 on the medical image data 112, weak annotations 114, and if available, the propagated annotations generated from interpolation and the one or more precise annotations 116.

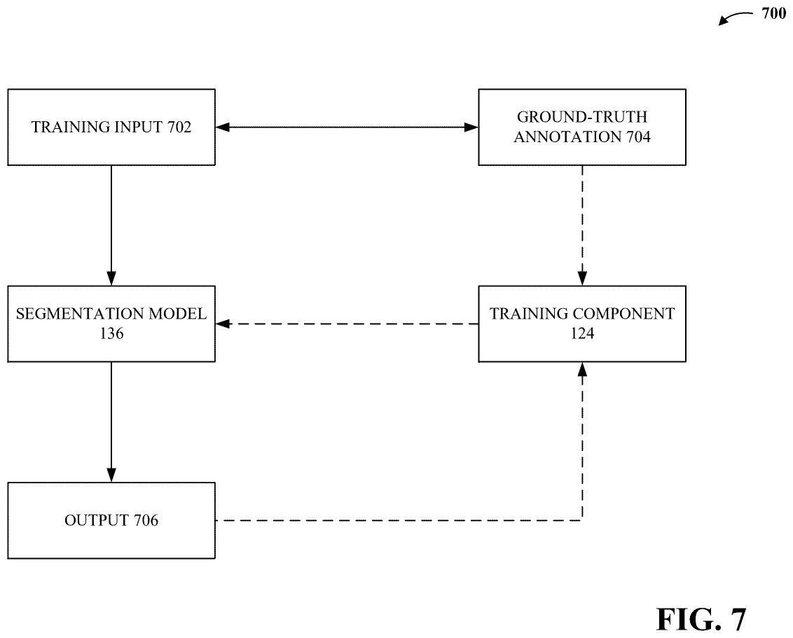

In various embodiments, training component 124 can electronically execute the segmentation model 130 on the training dataset 132, and such execution can cause segmentation model 130 to produce a set of pseudo labels. In various aspects, training component 124 can, as described herein, iteratively generate the set of pseudo labels and iteratively train the segmentation model using the set of pseudo labels. In this manner, training component 124 can iteratively expand the training dataset 132 by generating more annotations (e.g., pseudo labels) in each training iteration. Various non-limiting aspects are further described with respect to FIG. 7.

In various embodiments, the training component 124 can iteratively train the segmentation model 130 until the segmentation model 130 achieves a segmentation accuracy 134. In various aspects, the segmentation accuracy 134 can be any suitable performance metric or metrics indicative of the correspondence between predicted segmentation outputs and reference annotations. The performance metrics can include, but are not limited to, Dice similarity coefficient, 95th percentile Hausdorff Distance (HD95), Intersection-over-Union (IoU), pixel-wise accuracy, sensitivity, specificity, precision, recall, F1 score, boundary distance metrics, or combinations thereof. In some embodiments, the segmentation accuracy 134 can correspond to a predetermined threshold value, a dynamically adjusted threshold value, or a convergence criterion determined based on validation dataset performance. For example, the predetermined threshold values can be selected based on user input, clinical requirements, regulatory standards, empirical evaluation, prior experimental results, or application-specific performance constraints. In various instances, the training component 124 can terminate, pause, or modify training of the segmentation model 130 upon determining that the segmentation model 130 meets, satisfies, or exceeds the predetermined segmentation accuracy 134. For example, predetermined segmentation accuracy 134 can define corresponding threshold values for one or more performance metrics. Accordingly, training component 124 can iteratively train the segmentation model 130 on the set of pseudo labels until segmentation model 130 achieves values of the one or more performance metrics that are greater than or equal to the corresponding threshold values.

Thereafter, in various embodiments, the model component 126 can electronically execute the segmentation model 130 on an inputted medical image, and such execution can cause the segmentation model 130 to produce as output a segmented image for the inputted medical image. In some instances, model component 126 can render, on any suitable electronic display (e.g., computer screen, computer monitor, graphical user-interface), the segmented image, thereby enabling a user, technician, or medical professional can visually inspect or view the segmented image, which can aid the user, technician, or medical professional in making a diagnosis or prognosis. In various cases, the model component 126 can electronically transmit the segmented image to any suitable computing device.

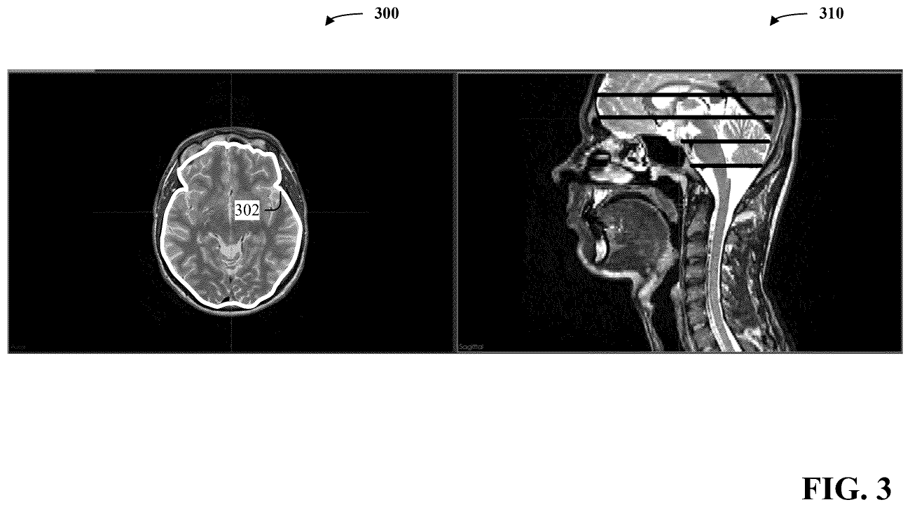

FIGS. 2 and 3 provide example, non-limiting medical image data with weak annotations in accordance with one or more embodiments described herein.

As a non-limiting example, medical image data 112 can comprise an MRI image 200, an MRI image 210, an MRI image 300, and an MRI image 310. FIG. 2 illustrates MRI image 200 with a first incomplete weak annotation and an MRI image 210 with a second incomplete weak annotations, wherein MRI image 200 and MRI image 210 depict a head region for brain segmentation. As shown, the incomplete weak annotation in MRI image 200 can comprise a two-dimensional bounding box 202. The two-dimensional bounding box 202 can coarsely identify a region containing a target anatomical structure without delineating precise structural. In this non-limiting example, two-dimensional bounding box 202 identifies a region containing a brain structure. As shown, the incomplete weak annotation in MRI image 210 can further comprise point annotations in addition to two-dimensional bounding box 202. In various aspects, the point annotations, such as point annotation 204, can comprise inner or outer labels. Specifically, the inner labels can indicate that the point is within the brain structure (depicted by gray circles), and the outer labels can indicate that the point is outside the brain structure (depicted by white circles). In other words, the inner labels can indicate regions that are desirable to segment, and outer labels can indicate regions that are not desirable to segment. For instance, point annotation 204 comprises an outer label, indicating that it is outside the brain structure and is not desirable to segment.

FIG. 3 illustrates slice-based annotations in an MRI image 300 and an MRI image 310. Specifically, MRI image 300 is an axial slice of the head region and comprises a complete annotation 302 of the brain structure. MRI image 310 depicts a lateral side view of the head region and comprises slice location annotations (depicted by vertical black lines), wherein MRI image 300 corresponds to one of the slices illustrated in MRI image 310.

As depicted in FIG. 2, the weak annotations in MRI image 200 and MRI image 210 are incomplete annotations. That is, there are regions in MRI image 200 and MRI image 210 that are unannotated or unlabeled. For example, regions outside of two-dimensional bounding box 202 are unannotated in MRI image 200 and MRI image 210. Similarly, as depicted in FIG. 3, the weak annotations in MRI image 300 and MRI image 310 are incomplete annotations. That is, besides the slice depicted in MRI image 300, the other slices in MRI image 310 are unannotated or unlabeled.

Although the weak annotations are incomplete weak annotations, the training component 124 can, as described herein, train segmentation model 130 on the incomplete weak annotations to generate a set of pseudo labels, wherein the segmentation model 130 can be subsequently iteratively trained using the incomplete weak annotations and the set of pseudo labels. Thus, annotation creation can be accelerated for training. For example, in various cases, there can be hundreds of slices in medical image data, such as in larger organs or anatomical structures (e.g., the brain). Accordingly, the weakly supervised training described herein can train the segmentation model 130 using incomplete weak annotations. That is, only a subset of the slices can be annotated, significantly reducing time consumption for annotation creation in the presence of numerous slices. Conversely, existing training techniques rely on precise annotations for a large portion of the slices, which can consume an extensive amount of time to manually annotate.

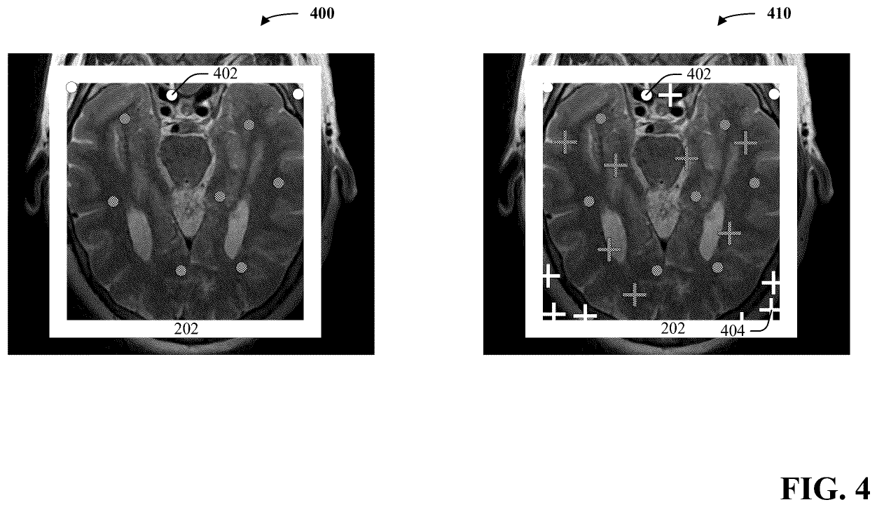

FIG. 4 illustrates example, non-limiting medical image data 400 showing how weak annotations can be propagated to unannotated regions of the medical image data in accordance with one or more embodiments described herein.

Various aspects are described with respect to FIGS. 2 and 3. In various instances, annotation generation component 122 can apply an interpolation technique to propagate the weak annotations 114 to unannotated regions of medical image data 112. For instance, annotation generation component 122 can apply an interpolation technique to propagate the weak annotations in MRI image 200, MRI image 210, MRI image 300, and MRI image 310 to unannotated regions of medical image data 112.

As a non-limiting example, annotation generation component 122 can propagate weak annotations from the slice of MRI image 300 to the other slices shown in MRI image 310. Specifically, annotation generation component 122 can, via the interpolation technique, copy points from a slice to adjacent slices. For instance, in medical image data 400, the point annotations, such as point annotation 402, can be propagated from an adjacent slice, wherein the point annotations comprise inner labels or outer labels. Similarly, as shown in medical image data 400, the two-dimensional bounding box 202 can be propagated onto the slice from MRI image 200 and 210.

As another non-limiting example, the point annotations in medical image data 400 can be propagated to unannotated regions within medical image data 400, resulting in MRI image 410. For instance, the point annotations in medical image data 400 (depicted by gray and white circles) can be propagated to generate additional point annotations (depicted by gray and white crosses), such as point annotation 404, that can comprise inner labels or outer labels.

Thereafter, training component 124 can train segmentation model 130 on the weak annotations from MRI image 200, MRI image 210, MRI image 300, MRI image 310, medical image data 400, and MRI image 410. That is, the training component 124 can train segmentation model 130 using the two-dimensional bounding box 202, the point annotations (e.g., point annotation 204, point annotation 402, point annotation 404), complete annotation 302, and other weak annotations collected for the medical image data (e.g., from weak annotations 114 or annotations propagated from weak annotations 114). As stated previously, propagating the weak annotations 114 to unannotated regions of medical image data 112 via an interpolation technique does not incur manual annotation time.

Then, training component 124 can execute the segmentation model 130 on the medical image data 112 to generate a set of pseudo labels. The pseudo labels and the weak annotations can then be utilized to iteratively train the segmentation model 130, wherein additional pseudo labels are generated at each training iteration and used in the next training iteration. This iterative process can be repeated until segmentation model 130 satisfies or exceeds the segmentation accuracy 134.

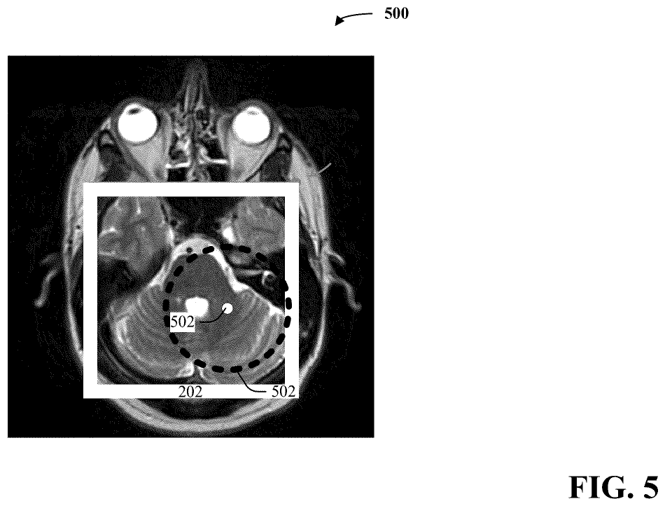

FIG. 5 illustrates an example, non-limiting medical image 500 showing how weak annotations can be propagated to unannotated regions of the medical image in accordance with one or more embodiments described herein.

In various embodiments, the annotation generation component 122 can propagate the weak annotations 114 within a predefined radius. More specifically, the annotation generation component 122 can generate the propagated annotations for unannotated regions of the medical image data 112 that are within the predefined radius from a weak annotation (e.g., within a defined distance from a point annotation). In various aspects, the predefined radius can be determined based on at least one of a predefined parameter, anatomical characteristics of a target structure, image resolution, or user input.

As a non-limiting example, as shown in medical image 500, the annotation generation component 122 can propagate a point annotation 502 within a region 504, where region 504 includes the area within a predefined radius from point annotation 502 (e.g., within two millimeters). Accordingly, the additional annotations generated from such propagation will be assigned to regions of the medical image 500 that are within region 504.

In various cases, annotation generation component 122 can apply the interpolation technique after each training iteration. That is, annotation generation component 122 can apply the interpolation technique to propagate the weak annotations 114 and the set of pseudo labels generated from previous iterations to the unannotated regions of the medical image data 112. In such instances, annotation generation component 122 can adjust the predefined radius for each iteration of propagation.

Although such propagation of weak annotations is primarily described as applying to point-based annotations, the propagation can be applied to any suitable types of annotations.

Furthermore, the annotation generation component 122 can select a size or thickness of the weak annotations 114 and/or the propagated annotations generated from propagating the weak annotations. Particularly, annotation generation component 122 can select a size or thickness of the weak annotations 114 and/or the propagated annotations by determining a spatial extent over which labels are applied. As a non-limiting example, annotation generation component 122 can adjust a contour thickness of bounding boxes. As another non-limiting example, annotation generation component 122 can adjust a radius of point-based annotations. As yet another non-limiting example, annotation generation component 122 can adjust a contour thickness of scribble-based annotations. As still another non-limiting example, annotation generation component 122 can adjust a thickness of slice-based annotations.

In other instances, the annotation generation component 122 can select a number of slices to propagate the weak annotations 114 to. As a non-limiting example, the annotation generation component 122 can adjust how many consecutive adjacent slices the weak annotations 114 will be propagated to.

In various instances, the size or thickness can be selected based on user input. As a non-limiting example, a user can select the size or thickness based on the target anatomical structure to be segmented. As another non-limiting example, the size or thickness can be dynamically selected based on empirical evaluation or prior experimental results.

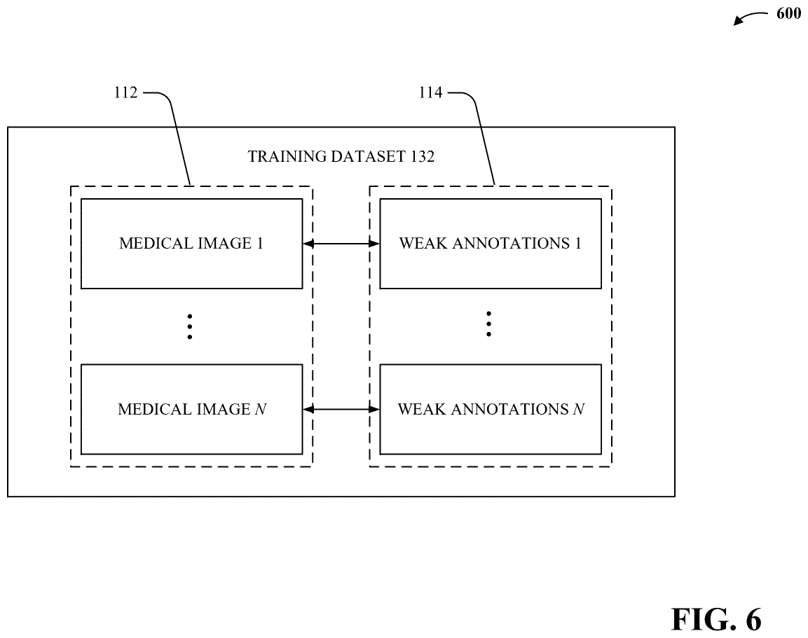

FIG. 6 illustrates an example, non-limiting block diagram 600 of a training dataset comprising weak annotations for training a segmentation model in accordance with one or more embodiments described herein.

In various aspects, the training component 124 can construct the training dataset 132 from medical image data 112 and weak annotations 114. In various instances, the training dataset 132 can further comprise the one or more precise annotations 116 (not shown).

As shown, the training dataset 132 can comprise medical image data 112. That is, medical image data 112 can comprise n medical images for any suitable positive integer n: a medical image 112(1) to a medical image 112(n). In various cases, the medical images can have any suitable format, size, or dimensionality.

Further, the training dataset 132 can comprise weak annotations 114. In various instances, the weak annotations 114 can correspond to the medical images of medical image data 112. Accordingly, weak annotations 114 can comprise n sets of weak annotations: a set of weak annotations 114(1) to a set of weak annotations 114(n). That is, each medical image of medical image data 112 can correspond to (e.g., in one-to-one fashion) a set of weak annotations.

In various aspects, each set of weak annotations can be considered as indicating or representing locations, classifications, or descriptive labels associated with a corresponding medical image that is known or otherwise deemed to be approximately correct or accurate. As a non-limiting example, the set of weak annotations 114(1) can correspond to the medical image 112(1). Accordingly, the set of weak annotations 114(1) can be considered as the known approximately correct or accurate locations, classifications, or descriptive labels associated with the medical image 112(1). As another non-limiting example, the set of weak annotations 114(n) can correspond to the medical image 112(n). So, the set of weak annotations 114(n) can be considered as the known approximately correct or accurate locations, classifications, or descriptive labels of the medical image 112(n). For instance, a set of weak annotations 114(i), for any positive integer isn, can comprise a bounding box with point annotations that approximately indicate or delineate a brain in a medical image 112(i).

Now, consider FIG. 7. FIG. 7 illustrates an example, non-limiting block diagram 700 showing how segmentation model 130 can be trained in accordance with one or more embodiments described herein.

In various aspects, the training component 124 can, prior to beginning training, initialize in any suitable fashion (e.g., random initialization) the trainable internal parameters (e.g., convolutional kernels, weight matrices, bias values) of the segmentation model 130.

In various aspects, the training component 124 can select, from the training dataset 132, a training input 702 and a ground-truth annotation 704 corresponding to the training input 702. Specifically, the training component 124 can select a medical image from medical image data 112 as training input 702. Further, the training component 124 can select a set of weak annotations from weak annotations 114 that corresponds to the medical image as the ground-truth annotation 704. In some cases, if one or more precise annotations 116 are available, the training component 124 can additionally select the precise annotations that correspond to the medical image as ground-truth annotation 704. In various instances, the training component 124 can execute the segmentation model 130 on the training input 702, thereby causing the segmentation model 130 to produce an output 706. More specifically, in some cases, an input layer of the segmentation model 130 can receive the training input 702, the training input 702 can complete a forward pass through one or more hidden layers of the segmentation model 130, and an output layer of the segmentation model 130 can compute the output 706 based on activation maps or intermediate features provided by the one or more hidden layers.

In various aspects, the output 706 can be considered as the predicted or inferred annotations or labels (e.g., as the predicted/inferred point-based annotations, the predicted/inferred slice-based annotations, the predicted/inferred scribble-based annotations, the predicted/inferred bounding boxes, the predicted/inferred segmentation mask that indicates a target anatomical structure) that the segmentation model 130 believes should correspond to the training input 702. In contrast, the ground-truth annotation 704 can be considered as the correct/accurate image comparison features and positioning quality (e.g., as the correct/accurate skin lines, the correct/accurate missing tissue, the correct/accurate positioning scores, the correct/accurate comparison metrics) that is known or deemed to correspond to the training input 702. In various aspects, the predicted or inferred annotations or labels can be considered pseudo labels (e.g., pseudo labels 802) that can be used to iteratively train the segmentation model 130. Note that, if the segmentation model 130 has so far undergone no or little training, then the output 706 can be highly inaccurate. In other words, the output 706 can be very different from the ground-truth annotation 704. Accordingly, the training component 124 can iteratively train the segmentation model 130 on the pseudo labels.

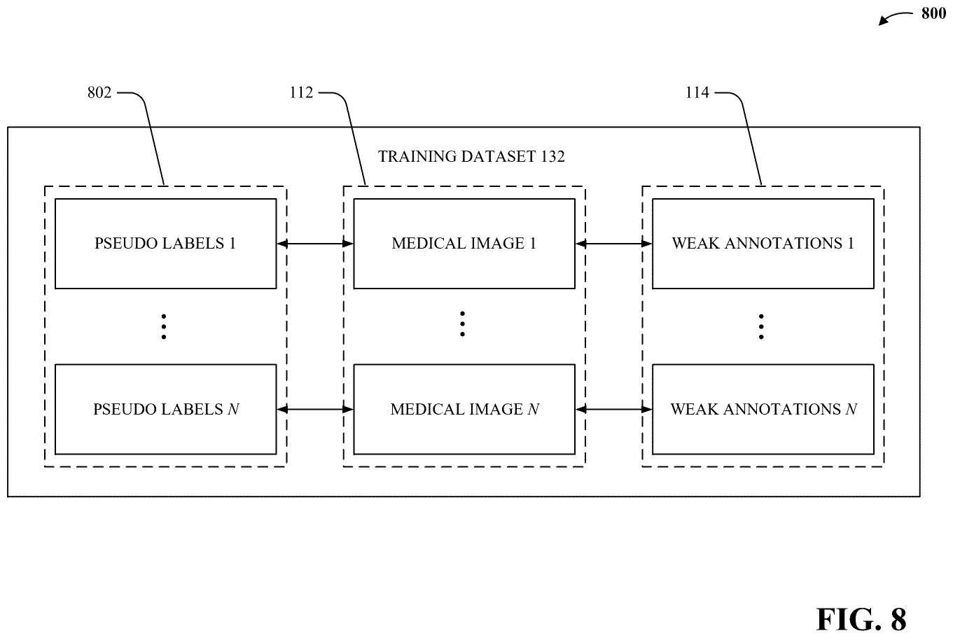

That is, the pseudo labels generated from a first training iteration can be used with the medical image data 112 and weak annotations 114 to train the segmentation model 130 in the next iteration, wherein the next iteration produces another set of pseudo labels. Accordingly, the next set of pseudo labels can be used with the medical image data 112, weak annotations 114, and previously generated pseudo labels to train the segmentation model 130 in the subsequent iteration. In this iterative training procedure, the segmentation model 130 can iteratively generate new training data (e.g., the pseudo labels) for iterative refinement of the segmentation model 130 until the segmentation accuracy is achieved. In other words, the pseudo labels from a previous iteration can be used as additional ground-truth annotations in a subsequent iteration. Various aspects are further described with respect to FIG. 8.

In various aspects, the training component 124 can compute one or more errors or losses (e.g., MAE, MSE, cross-entropy) between the output 706 and the ground-truth annotation 704. In particular, the training component 124 can compute the one or more errors or losses such that unannotated regions of the medical image data 112 are excluded from the computation. Therefore, the unannotated regions do not require ground-truth annotations. In various aspects, the training component 124 can employ any suitable loss function that excludes the unannotated regions of the medical image data 112 to compute the one or more errors or losses between the output 706 and the ground-truth annotation 704. The computational complexity to compute the one or more errors or losses in this manner is similar to that of training with precise masks. In any case, the training component 124 can incrementally update, via backpropagation, the trainable internal parameters of the segmentation model 130, based on such one or more errors or losses.

In various cases, the training component 124 can repeat such execution-and-update procedure for each training input in the training dataset 132. This can ultimately cause the trainable internal parameters of the segmentation model 130 to become iteratively optimized for accurately determining annotations or labels (e.g., accurately generating segmentation masks that indicates a target anatomical structure) of inputted medical image data. In various aspects, the training component 124 can implement any suitable training batch sizes, any suitable error/loss functions, or any suitable training termination criteria. For instance, the training component 124 can terminate training in response to the segmentation model 130 satisfying or exceeding the segmentation accuracy 134. In other instances, the training component 124 can terminate training in response to annotating all regions in medical image data 112. In still other instances, the training component 124 can terminate training in response to the segmentation accuracy not improving upon the segmentation accuracy from previous training iterations.

In various cases, the training component 124 can train the segmentation model 130 on one or more training datasets comprising medical image data and corresponding weak annotations. For example, in various aspects, the training component 124 can initially train the segmentation model 130 on medical image data 112 and weak annotations 114 from training dataset 132. Thereafter, the training component 124 can train the segmentation model 130 on another training dataset to iteratively generate the pseudo labels for iterative retraining and tuning of segmentation model 130. That is, the training component 124 can generate the pseudo labels for additional sets of medical image data to facilitate the iterative retraining and tuning of segmentation model 130.

FIG. 8 illustrates an example, non-limiting block diagram 800 of a training dataset comprising weak annotations and pseudo labels for training a segmentation model in accordance with one or more embodiments described herein.

In various embodiments, following the initial training of segmentation model 130 on medical image data 112 and weak annotations 114 from training dataset 132, the training component 124 can generate a set of pseudo labels 802 for unannotated regions of medical image data 112 via the segmentation model. Specifically, the training component 124 can execute the segmentation model 130 on the medical image data 112, thereby causing the segmentation model 130 to produce the set of pseudo labels 802 for unannotated regions of medical image data 112. In various embodiments, the segmentation model 130 can produce the set of pseudo labels 802 for a subset of unannotated regions of medical image data 112. In various cases, the subset of unannotated regions can be selected based on spatial characteristics of medical image data 112. For example, the subset of unannotated regions can be defined as unannotated regions located within a predefined radius of one or more data points in medical image data 112. In various embodiments, the one or more data points can be sampled from spatial coordinates corresponding to the image domain, including pixel locations in a two-dimensional image or voxel locations in a three-dimensional image volume. The radius can correspond to a fixed distance parameter expressed in pixels, voxels, or physical units (e.g., millimeters), and can define a circular, spherical, or otherwise bounded neighborhood surrounding each data point. All unannotated regions that fall within the defined neighborhood can be selected for pseudo label generation, while unannotated regions outside the neighborhood can remain excluded during a given training iteration. In various embodiments, the radius value can be static across training iterations, whereas in other embodiments the radius can be dynamically adjusted based on model confidence, training progression, or dataset characteristics (e.g., the radius can be incrementally increased each training iteration).

In various aspects, the training component 124 can add the set of pseudo labels 802 to the training dataset 132. Thereafter, the training component 124 can retrain the segmentation model 130 on the training dataset 132, which now comprises the set of pseudo labels 802 in addition to medical image data 112 and weak annotations 114.

In various instances, the set of pseudo labels 802 can correspond to the medical images of medical image data 112. Accordingly, set of pseudo labels 802 can comprise n sets of pseudo labels: a set of pseudo labels 802(1) to a set of pseudo labels 802(n). That is, each medical image of medical image data 112 can correspond to (e.g., in one-to-one fashion) a set of pseudo labels.

In various aspects, each set of pseudo labels can be considered as indicating or representing locations, classifications, or descriptive labels associated with a corresponding medical image that is known or otherwise deemed to be approximately correct or accurate (e.g., as predicted by segmentation model 130 based on medical image data 112 and weak annotations 114). As a non-limiting example, the set of pseudo labels 802(1) can correspond to the medical image 112(1). Accordingly, the set of pseudo labels 802(1) can be considered as the known approximately correct or accurate locations, classifications, or descriptive labels associated with the medical image 112(1). As another non-limiting example, the set of pseudo labels 802(n) can correspond to the medical image 112(n). So, the set of pseudo labels 802(n) can be considered as the known approximately correct or accurate locations, classifications, or descriptive labels of the medical image 112(n).

Accordingly, the training component 124 can select, from the training dataset 132, a medical image from medical image data 112 as training input 702. Further, the training component 124 can select a set of pseudo labels from the set of pseudo labels 802 that corresponds to the medical image as the ground-truth annotation 704. In various instances, the training component 124 can execute the segmentation model 130 on the training input 702, thereby causing the segmentation model 130 to produce an output 706. More specifically, the output 706 can be another set of pseudo labels. Accordingly, the training component 124 can add the new set of pseudo labels to training dataset 132.

In various aspects, the training component 124 can compute the one or more errors or losses (e.g., MAE, MSE, cross-entropy) between the output 706 and the ground-truth annotation 704 (e.g., between the new set of pseudo labels and the set of pseudo labels generated from the previous execution), and the training component 124 can incrementally update, via backpropagation, the trainable internal parameters of the segmentation model 130, based on such one or more errors or losses.

In various cases, the training component 124 can repeat such execution-and-update procedure for each training input in the training dataset 132. Specifically, the training component 124 can iteratively generate additional pseudo labels, wherein the pseudo labels generated across successive iterations can be added to and retained in training dataset 132 for subsequent retraining iterations. This can ultimately cause the trainable internal parameters of the segmentation model 130 to become iteratively optimized for accurately determining annotations or labels (e.g., accurately generating segmentation masks that indicates a target anatomical structure) of inputted medical image data. Such execution-and-update procedure can be repeated until the segmentation accuracy of segmentation model 130 meets a predetermined threshold as defined by segmentation accuracy 134.

By iteratively generating the set of pseudo labels 802 for subsets of the unannotated regions of medical image data 112 and iteratively training the segmentation model 130 on the set of pseudo labels 802, the training component 124 can gradually expand the training dataset 132 to comprise more annotations for retraining and tuning the segmentation model 130. That is, such weakly supervised training can enable a gradual expansion of annotations or labels to the unannotated regions of medical image data 112 for training segmentation model 130.

In various cases, in response to generating pseudo labels 802 (e.g., after one or more training iterations), the training component 124 can train a second segmentation model on training dataset 132. This can enable the second segmentation model to converge quicker than retraining the segmentation model 130. In such instances, the segmentation model 130 and the second segmentation model can form respective sub-models of a single segmentation model.

The second segmentation model can comprise a similar internal architecture to that of segmentation model 130 and can be trained in similar fashion to segmentation model 130, except that the initial training on only medical image data 112 and weak annotations 114 will be skipped. That is, the second segmentation model can be initially trained on the medical image data 112, weak annotations 114, and the pseudo labels 802 generated by segmentation model 130. In other words, the training component 124 can train segmentation model 130 on the medical image data 112 and weak annotations 114 to generate a larger training dataset for training the second segmentation model.

Initially training the second segmentation model on a larger dataset rather than retraining or tuning the segmentation model 130 with the larger dataset can improve convergence by enabling the second segmentation model to learn generalizable feature representations and by initializing model parameters closer to an optimal solution. The larger dataset can also provide more statistically stable gradient estimates, which can improve optimization stability and reduce training variability. As a result, subsequent training or fine-tuning of the second segmentation model can converge more quickly and reliably than retraining or tuning the segmentation model 130 with the larger dataset.

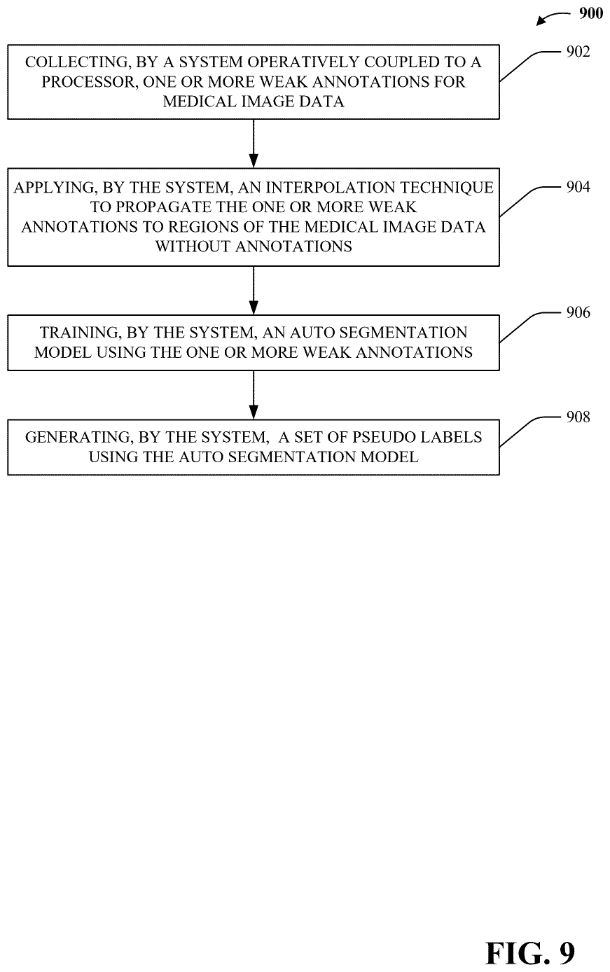

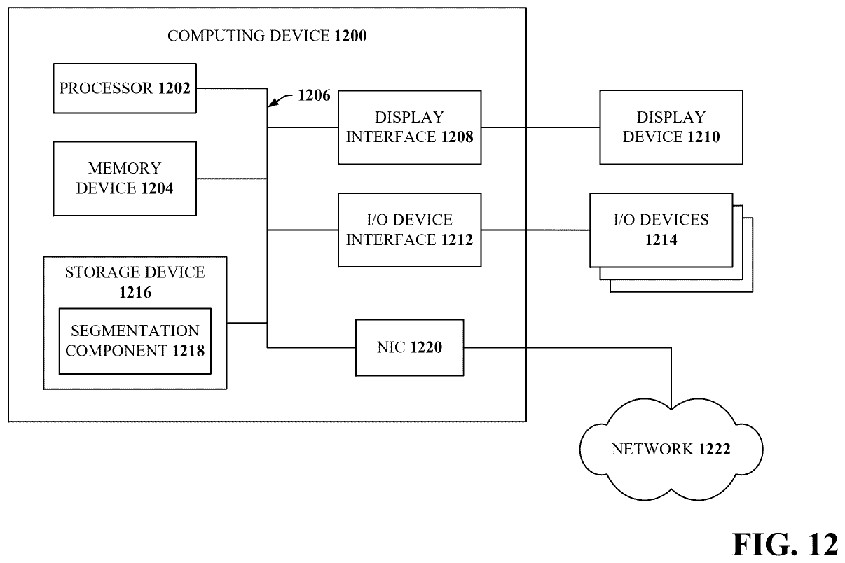

FIG. 9 illustrates a flow diagram of an example, non-limiting computer-implemented method 900 that can facilitate weakly supervised segmentation of medical image data in accordance with one or more embodiments described herein. In various cases, the weakly supervised image segmentation system 102 can facilitate the computer-implemented method 900. In some examples, the computer-implemented method 900 can be implemented with the computing device 1200 described below in relation to FIG. 12.

At block 902, the computer-implemented method 900 can collecting, by a system (e.g., via access component 120) operatively coupled to a processor (e.g., 106), one or more weak annotations (e.g., 114) for medical image data (e.g., 112).

At block 904, the computer-implemented method 900 can include applying, by the system (e.g., via annotation generation component 122), an interpolation technique to propagate the one or more weak annotations to regions of the medical image data without annotations.

At block 906, the computer-implemented method 900 can include training, by the system (e.g., via training component 124), a segmentation model (e.g., 136) using the one or more weak annotations.

At block 908, the computer-implemented method 900 can include generating, by the system (e.g., via training component 124), a set of pseudo labels (e.g., 802) using the segmentation model.

In some examples, techniques herein can be used for fine retraining or tuning segmentation models. In some examples, blocks 906 and 908 can be repeated so that the computer-implemented method 900 can include training the segmentation model (e.g., on the one or more weak annotations or the set of pseudo labels) and generating the set of pseudo labels until a segmentation accuracy meets a predetermined threshold (e.g., 134).

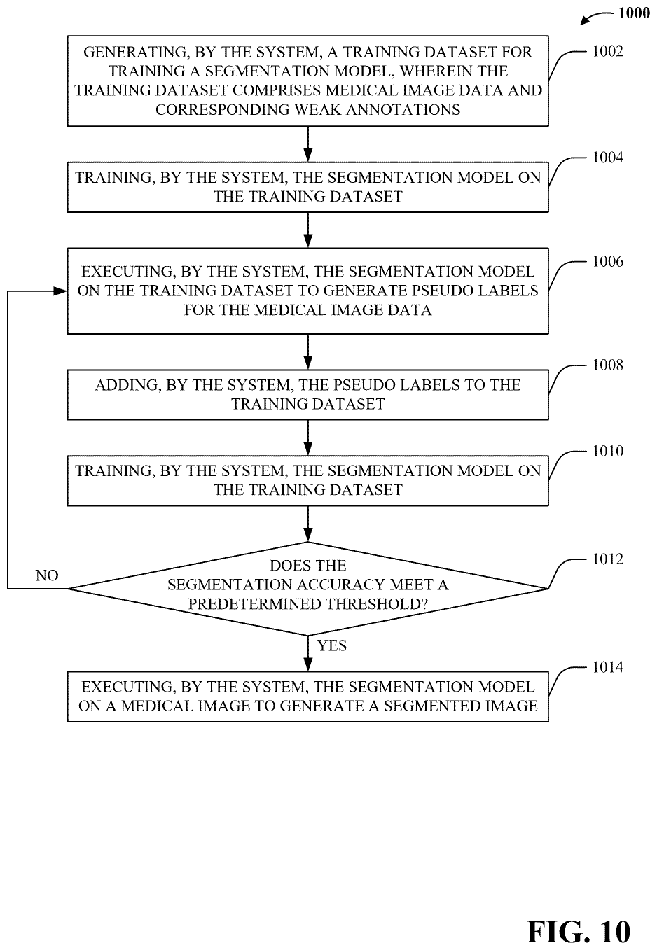

FIG. 10 illustrates a flow diagram of an example, non-limiting computer-implemented method 1000 that can facilitate multi-modal retrieval-augmented image segmentation in accordance with one or more embodiments described herein. In various cases, the weakly supervised image segmentation system 102 can facilitate the computer-implemented method 1000. In some examples, the computer-implemented method 1000 can be implemented with the computing device 1200 described below in relation to FIG. 12.

At block 1002, the computer-implemented method 1000 can generating, by a system (e.g., via annotation generation component 122) operatively coupled to a processor (e.g., 106), a training dataset (e.g., 132) for training a segmentation model (e.g., 136), wherein the training dataset comprises medical image data (e.g., 112) and corresponding weak annotations (e.g., 114).

At block 1004, the computer-implemented method 1000 can include training, by the system (e.g., via training component 124), the segmentation model on the training dataset.

At block 1006, the computer-implemented method 1000 can include executing, by the system (e.g., via training component 124), the segmentation model on the training dataset to generate a set of pseudo labels (e.g., 802) for the medical image data.

At block 1008, the computer-implemented method 1000 can include adding, by the system (e.g., via training component 124), the set of pseudo labels to the training dataset.

At block 1010, the computer-implemented method 1000 can include training, by the system (e.g., via training component 124), the segmentation model on the training dataset.

At block 1012, the computer-implemented method 1000 can include determining whether a segmentation accuracy of the segmentation model meets a predetermined threshold. If yes, computer-implemented method 1000 can proceed to block 1014. If no, computer-implemented method 1000 can proceed back to block 1006.

At block 1014, the computer-implemented method 1000 can include generating, by the system (e.g., via model component 126), the segmentation model on a medical image to generate a segmented image.

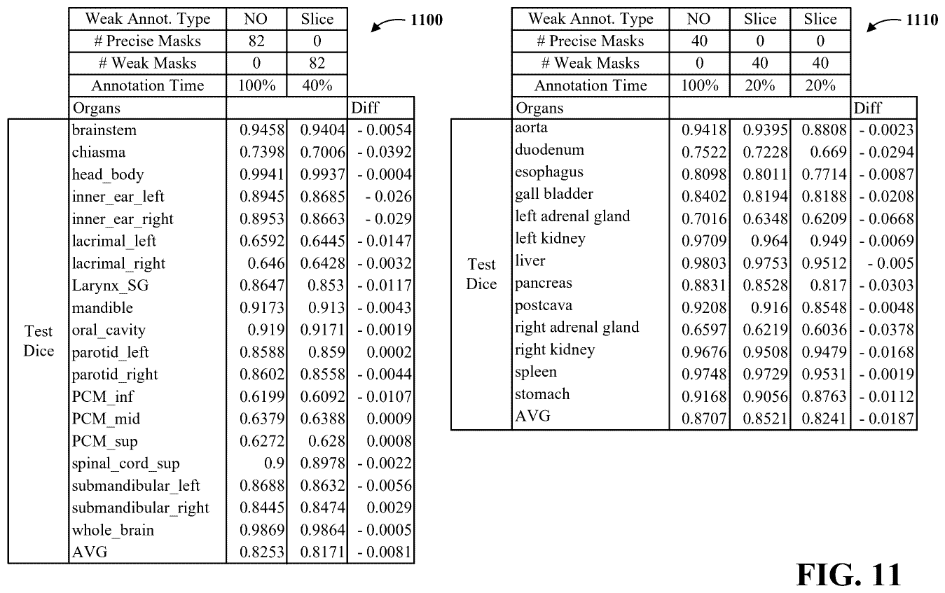

FIG. 11 illustrates charts 1100 and 1110 showing model performance of a segmentation model trained on weak annotations in accordance with one or more embodiments described herein.

First, consider Table 1 and Table 2, described below. Table 1 illustrates the performance of a weakly supervised-trained segmentation model (e.g., a segmentation model trained using the methods described herein) and a precise annotation-trained segmentation model (e.g., a segmentation model trained on only precise annotations) trained to segment MRI image data. Specifically, the segmentation models are trained to perform brain segmentation in MRI image data.

In this example implementation, the training dataset includes 100 volumetric MRI images, comprising 50 T1WI images and 50 T2WI images with precise brain segmentation masks. Further, the training dataset is split into two halves for training and testing.

The precise annotation-trained segmentation model is trained on 5 and 10 precise annotations (precise masks) and the weakly supervised-trained segmentation model is trained on 40 weak annotations (point-based annotations) with 2 precise annotations. The performance of each segmentation model is then evaluated based on test Dice and test HD95 metrics.

TABLE 1

| | | Weak annotation Type | | | | | | | |
|---|---|---|---|---|---|---|---|---|---|
| | | None | | | Point-Based | | | | |
| | | Point Threshold | | | | | | | |
| | | N/A | | | 1.5 | 3 | 6 | 12 | Inf |
| | | # Precise Masks | | | | | | | |
| | | 5 | 5 | 10 | 2 | | | | |
| | | # Weak Masks | | | | | | | |
| | | 0 | | | 40 | | | | |
| Test | T1 | 0.9749 | 0.8246 | 0.9766 | 0.9764 | 0.9784 | 0.9786 | 0.9789 | 0.9789 |
| Dice | T2 | 0.02 | 0.9891 | 0.9898 | 0.9861 | 0.9868 | 0.9873 | 0.9876 | 0.9875 |
| | Average | 0.4975 | 0.9068 | 0.9832 | 0.9813 | 0.9826 | 0.9829 | 0.9832 | 0.9832 |
| Test | T1 | 4.9 | 28.7 | 2.5 | 2.5 | 2.3 | 2.3 | 2.3 | 2.3 |
| HD95 | T2 | 103 | 1.8 | 1.8 | 2.2 | 2.1 | 2 | 2 | 2 |
| | Average | 53.9 | 15.2 | 2.1 | 2.4 | 2.2 | 2.2 | 2.1 | 2.1 |

As shown in Table 1, the weakly supervised-trained segmentation model achieves a higher accuracy than the precise annotation-trained segmentation model. Further, the time to create the weak annotations consumes approximately 20% of the annotation time for creating the precise annotations. For example, creating the 10 precise annotations takes approximately the same time as creating the 40 weak annotations and 2 precise annotations. Thus, as demonstrated by Table 1, the weak supervised training of segmentation models described herein can achieve an increased, or at least a comparable, accuracy in medical image segmentation while significantly reducing the time required to generate the annotated training dataset.

Table 2 illustrates the performance when the precise annotation-trained segmentation model is trained on 5 and 10 precise annotations and the weakly supervised-trained segmentation model is trained on 50 weak annotations (slice-based annotations) with 0 precise annotations, and on 40 weak annotations with 2 precise annotations. The performance of each segmentation model is evaluated based on test Dice and test HD95 metrics.

TABLE 2

| | | Weak annotation Type | | | | |
|---|---|---|---|---|---|---|
| | | None | | | Slice-Based | |
| | | # Precise Masks | | | | |
| | | 5 | 5 | 10 | 0 | 2 |
| | | # Weak Masks | | | | |
| | | 0 | | | 50 | 40 |
| Test | T1 | 0.9749 | 0.8246 | 0.9766 | 0.9805 | 0.9800 |
| Dice | T2 | 0.02 | 0.9891 | 0.9898 | 0.9900 | 0.9902 |
| | Average | 0.4975 | 0.9068 | 0.9832 | 0.9853 | 0.9851 |
| Test | T1 | 4.9 | 28.7 | 2.5 | 2.0 | 2.1 |
| HD95 | T2 | 103 | 1.8 | 1.8 | 1.8 | 1.7 |
| | Average | 53.9 | 15.2 | 2.1 | 1.9 | 1.9 |

As shown in Table 2, the weakly supervised-trained segmentation models achieve a higher accuracy than the precise annotation-trained segmentation model. Additionally, the weakly supervised-trained segmentation models achieve a consistent accuracy for T1W1 images and T2W1 images. Further, the annotation time to create the weak annotations is approximately five times faster than the annotation time to create the precise annotations. For example, 50 weak annotations can be created in approximately the same time as 5 precise annotations. Thus, as demonstrated by Table 2, the embodiments described herein can achieve an increased, or at least a comparable, accuracy in medical image segmentation across different imaging sequences while significantly reducing the time required to generate the annotated training dataset.

Table 1 and Table 2 therefore demonstrate that the weakly supervised segmentation described herein can accurately segment medical image data of different protocols or sequences by utilizing primarily weak annotations instead of precise annotations, wherein the time to create the weak annotations is significantly less than time to create the precise annotations. In other words, the embodiments herein can accelerate the annotation of training data for segmentation models. Thus, more medical image data of different protocols or sequences can be annotated and used to train the segmentation model to accurately segment medical image data.

Turning to FIG. 11, chart 1100 and chart 1110 depict model performance of the segmentation model on other anatomical structures. That is, the segmentation model described by Table 1 and Table 2 was trained for brain segmentation. In chart 1100, the segmentation model was trained for segmentation of various anatomical structures in the head and neck region. In chart 1110, the segmentation model was trained for segmentation of various anatomical structures in the abdominal region.

Chart 1100 and chart 1110 illustrate the difference (denoted by “Diff” in FIG. 11) in performance between training on only precise annotations and the weakly supervised training described herein.

For the head and neck region, a weakly supervised-trained segmentation model was trained on 82 slice-based weak annotations and 0 precise annotations, whereas the other segmentation model was trained on 82 precise annotations. For the abdominal region, a first weakly supervised-trained segmentation model was trained on 40 point-based weak annotations and 0 precise annotations, a second weakly supervised-trained segmentation model was trained on 40 slice-based weak annotations and 0 precise annotations, and the other segmentation model was trained on 40 precise annotations.

As shown in chart 1100 and chart 1110, the weakly supervised-trained segmentation models achieve a performance comparable to precise annotation-trained segmentation models while significantly reducing the annotation time. Specifically, for the head and neck region, the weakly supervised-trained segmentation model achieves a comparable performance while requiring only approximately 40% of the annotation time for the precise annotation-trained segmentation model. Further, for the abdominal region, the weakly supervised-trained segmentation model achieves a comparable performance while requiring only approximately 20% of the annotation time for the precise annotation-trained segmentation model.

Accordingly, Table 1, Table 2, and FIG. 11 demonstrate the improvements in medical image segmentation resulting from the weakly supervised training described herein.

Note that, in various instances, access component 120, annotation generation component 122, training component 124, and/or model component 126 can collectively be considered as being one or more software components of weakly supervised image segmentation system 102. In various aspects, it should be appreciated that the one or more software components are described primarily herein as comprising four components (e.g., access component 120, annotation generation component 122, training component 124, and model component 126) for ease of explanation and illustration. However, the one or more software components are not limited to being implemented as exactly such four components in every embodiment. Indeed, in some embodiments, the functionalities described herein of such four components can be combined in any suitable fashions, so as to be implemented in or by fewer than four components (e.g., in some cases, a single component can perform all of the functionalities that are described herein with respect to the access component 120, the annotation generation component 122, the training component 124, and the model component 126). In other embodiments, the functionalities described herein of such four components can instead be distributed, separated, split, or fragmented in any suitable fashions, so as to be implemented in or by more than four components (e.g., two or more components can facilitate the functionalities that are performable by the access component 120; two or more components can facilitate the functionalities that are performable by the annotation generation component 122; two or more components can facilitate the functionalities that are performable by the training component 124; and two or more components can facilitate the functionalities that are performable by the model component 126).

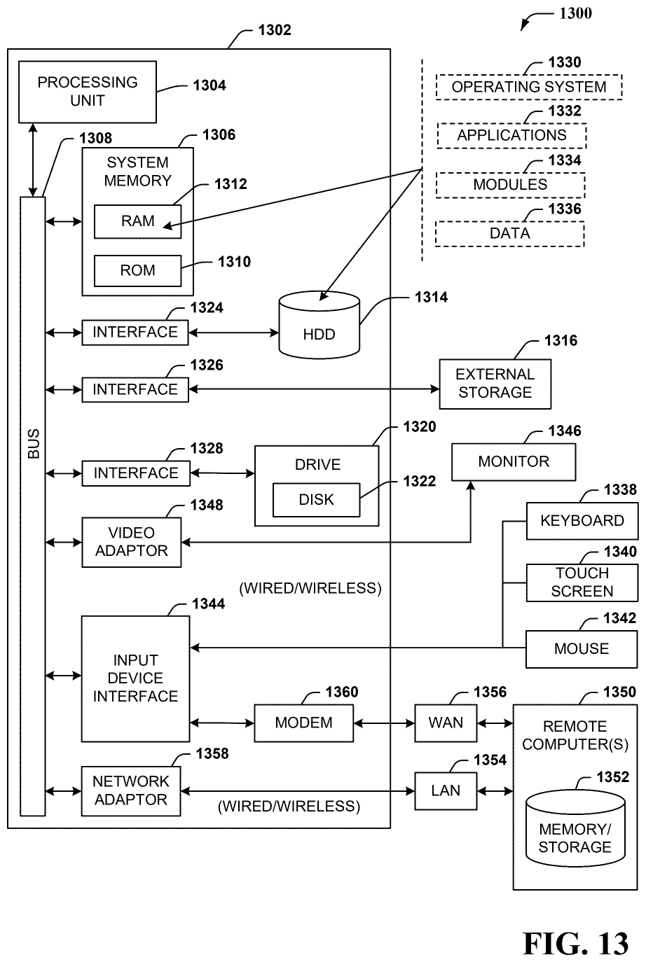

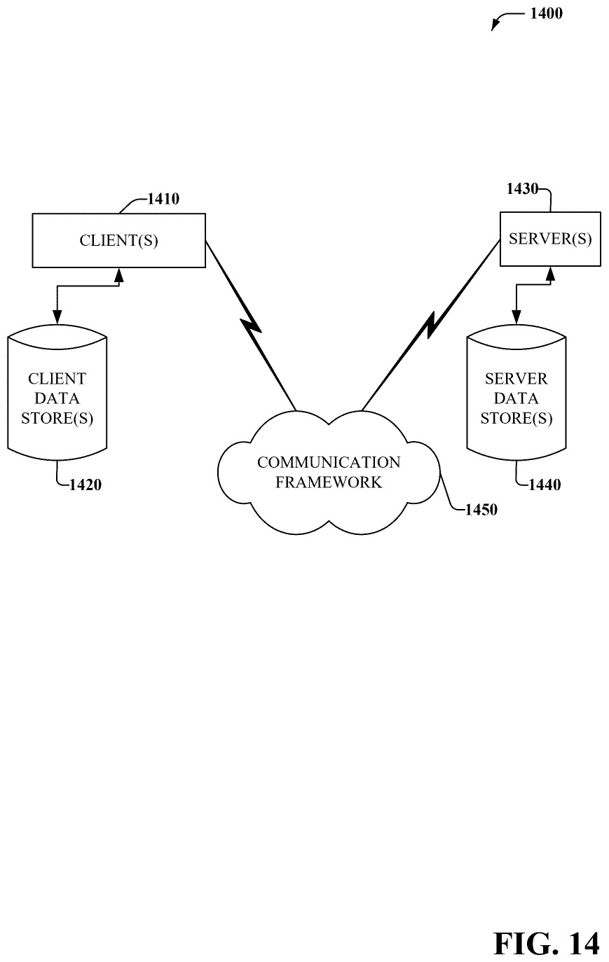

FIG. 12 is a block diagram of an example of a computing device that can segment medical image data. The computing device 1200 may be, for example, a server, a laptop computer, a desktop computer, a tablet computer, or a mobile phone, among others. The computing device 1200 may include a processor 1202 that is adapted to execute stored instructions, as well as a memory device 1204 that stores instructions that are executable by the processor 1202. The processor 1202 can be a single-core processor, a multi-core processor, a computing cluster, or any number of other configurations. The memory device 1204 can include random access memory, read-only memory, flash memory, or any other suitable memory systems. The instructions that are executed by the processor 1202 may be used to implement a method that can segment medical image data, as described in greater detail above in relation to FIGS. 1-10 and below in relation to FIGS. 13 and 14.

The processor 1202 may also be linked through the system interconnect 1206 (e.g., PCI, PCI-Express, NuBus, etc.) to a display interface 1208 adapted to connect the computing device 1200 to a display device 1210. The display device 1210 may include a display screen that is a built-in component of the computing device 1200. The display device 1210 may also include a computer monitor, television, or projector, among others, which is externally connected to the computing device 1200. The display device 1210 can include light-emitting diodes (LEDs), micro-LEDs, Organic light-emitting diode (OLED) displays, among others.

The processor 1202 may be connected through a system interconnect 1206 to an input/output (I/O) device interface 1212 adapted to connect the computing device 1200 to one or more I/O devices 1214. The I/O devices 1214 may include, for example, a keyboard and a pointing device, wherein the pointing device may include a touchpad or a touchscreen, among others. The I/O devices 1214 may be built-in components of the computing device 1200 or may be devices that are externally connected to the computing device 1200.

In some embodiments, the processor 1202 may also be linked through the system interconnect 1206 to a storage device 1216 that can include a hard drive, an optical drive, a USB flash drive, an array of drives, or any combinations thereof. In some embodiments, the storage device 1216 can include any suitable applications. In some embodiments, the storage device 1216 can include segmentation component 110. In some embodiments, the segmentation component 1218 can perform any segmentation operations described above in relation to FIGS. 1-11 (e.g., can implement the functionalities of the segmentation component 110).

In some examples, a network interface controller (also referred to herein as a NIC) 1220 may be adapted to connect the computing device 1200 through the system interconnect 1206 to a network 1222. The network 1222 may be a cellular network, a radio network, a wide area network (WAN), a local area network (LAN), or the Internet, among others. The network 1222 can enable data, such as alerts, among other data, to be transmitted from the computing device 1200 to remote computing devices, remote display devices, and the like.

It is to be understood that the block diagram of FIG. 12 is not intended to indicate that the computing device 1200 is to include all of the components shown in FIG. 12. Rather, the computing device 1200 can include fewer or additional components not illustrated in FIG. 12 (e.g., additional memory components, embedded controllers, additional modules, additional network interfaces, etc.). Furthermore, any of the functionalities of the segmentation component 110 may be partially, or entirely, implemented in hardware and/or in the processor 1202. For example, the functionality may be implemented with an application-specific integrated circuit, logic implemented in an embedded controller, or in logic implemented in the processor 1202, among others. In some embodiments, the functionalities of the segmentation component 110 can be implemented with logic, wherein the logic, as referred to herein, can include any suitable hardware (e.g., a processor, among others), software (e.g., an application, among others), firmware, or any suitable combination of hardware, software, and firmware.

In order to provide additional context for various embodiments described herein, FIG. 13 and the following discussion are intended to provide a brief, general description of a suitable computing environment 1300 in which the various embodiments of the embodiment described herein can be implemented. While the embodiments have been described above in the general context of computer-executable instructions that can run on one or more computers, those skilled in the art will recognize that the embodiments can be also implemented in combination with other program modules or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multi-processor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 13, the example environment 1300 for implementing various embodiments of the aspects described herein includes a computer 1302, the computer 1302 including a processing unit 1304, a system memory 1306 and a system bus 1308. The system bus 1308 couples system components including, but not limited to, the system memory 1306 to the processing unit 1304. The processing unit 1304 can be any of various commercially available processors. Dual microprocessors and other multi processor architectures can also be employed as the processing unit 1304.

The system bus 1308 can be any of several types of bus structure that can further interconnect to a memory bus (with or without a memory controller), a peripheral bus, and a local bus using any of a variety of commercially available bus architectures. The system memory 1306 includes ROM 1310 and RAM 1312. A basic input/output system (BIOS) can be stored in a non-volatile memory such as ROM, erasable programmable read only memory (EPROM), EEPROM, which BIOS contains the basic routines that help to transfer information between elements within the computer 1302, such as during startup. The RAM 1312 can also include a high-speed RAM such as static RAM for caching data.

The computer 1302 further includes an internal hard disk drive (HDD) 1314 (e.g., EIDE, SATA), one or more external storage devices 1316 (e.g., a magnetic floppy disk drive (FDD) 1316, a memory stick or flash drive reader, a memory card reader, etc.) and a drive 1320, e.g., such as a solid state drive, an optical disk drive, which can read or write from a disk 1322, such as a CD-ROM disc, a DVD, a BD, etc. Alternatively, where a solid state drive is involved, disk 1322 would not be included, unless separate. While the internal HDD 1314 is illustrated as located within the computer 1302, the internal HDD 1314 can also be configured for external use in a suitable chassis (not shown). Additionally, while not shown in environment 1300, a solid state drive (SSD) could be used in addition to, or in place of, an HDD 1314. The HDD 1314, external storage device(s) 1316 and drive 1320 can be connected to the system bus 1308 by an HDD interface 1324, an external storage interface 1326 and a drive interface 1328, respectively. The interface 1324 for external drive implementations can include at least one or both of Universal Serial Bus (USB) and Institute of Electrical and Electronics Engineers (IEEE) 1134 interface technologies. Other external drive connection technologies are within contemplation of the embodiments described herein.

The drives and their associated computer-readable storage media provide nonvolatile storage of data, data structures, computer-executable instructions, and so forth. For the computer 1302, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1312, including an operating system 1330, one or more application programs 1332, other program modules 1334 and program data 1336. All or portions of the operating system, applications, modules, or data can also be cached in the RAM 1312. The systems and methods described herein can be implemented utilizing various commercially available operating systems or combinations of operating systems.

Computer 1302 can optionally comprise emulation technologies. For example, a hypervisor (not shown) or other intermediary can emulate a hardware environment for operating system 1330, and the emulated hardware can optionally be different from the hardware illustrated in FIG. 13. In such an embodiment, operating system 1330 can comprise one virtual machine (VM) of multiple VMs hosted at computer 1302. Furthermore, operating system 1330 can provide runtime environments, such as the Java runtime environment or the.NET framework, for applications 1332. Runtime environments are consistent execution environments that allow applications 1332 to run on any operating system that includes the runtime environment. Similarly, operating system 1330 can support containers, and applications 1332 can be in the form of containers, which are lightweight, standalone, executable packages of software that include, e.g., code, runtime, system tools, system libraries and settings for an application.

Further, computer 1302 can be enable with a security module, such as a trusted processing module (TPM). For instance with a TPM, boot components hash next in time boot components, and wait for a match of results to secured values, before loading a next boot component. This process can take place at any layer in the code execution stack of computer 1302, e.g., applied at the application execution level or at the operating system (OS) kernel level, thereby enabling security at any level of code execution.

A user can enter commands and information into the computer 1302 through one or more wired/wireless input devices, e.g., a keyboard 1338, a touch screen 1340, and a pointing device, such as a mouse 1342. Other input devices (not shown) can include a microphone, an infrared (IR) remote control, a radio frequency (RF) remote control, or other remote control, a joystick, a virtual reality controller or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1304 through an input device interface 1344 that can be coupled to the system bus 1308, but can be connected by other interfaces, such as a parallel port, an IEEE 1134 serial port, a game port, a USB port, an IR interface, a BLUETOOTH® interface, etc.

A monitor 1346 or other type of display device can be also connected to the system bus 1308 via an interface, such as a video adapter 1348. In addition to the monitor 1346, a computer typically includes other peripheral output devices (not shown), such as speakers, printers, etc.

The computer 1302 can operate in a networked environment using logical connections via wired or wireless communications to one or more remote computers, such as a remote computer(s) 1350. The remote computer(s) 1350 can be a workstation, a server computer, a router, a personal computer, portable computer, microprocessor-based entertainment appliance, a peer device or other common network node, and typically includes many or all of the elements described relative to the computer 1302, although, for purposes of brevity, only a memory/storage device 1352 is illustrated. The logical connections depicted include wired/wireless connectivity to a local area network (LAN) 1354 or larger networks, e.g., a wide area network (WAN) 1356. Such LAN and WAN networking environments are commonplace in offices and companies, and facilitate enterprise-wide computer networks, such as intranets, all of which can connect to a global communications network, e.g., the Internet.

When used in a LAN networking environment, the computer 1302 can be connected to the local network 1354 through a wired or wireless communication network interface or adapter 1358. The adapter 1358 can facilitate wired or wireless communication to the LAN 1354, which can also include a wireless access point (AP) disposed thereon for communicating with the adapter 1358 in a wireless mode.

When used in a WAN networking environment, the computer 1302 can include a modem 1360 or can be connected to a communications server on the WAN 1356 via other means for establishing communications over the WAN 1356, such as by way of the Internet. The modem 1360, which can be internal or external and a wired or wireless device, can be connected to the system bus 1308 via the input device interface 1344. In a networked environment, program modules depicted relative to the computer 1302 or portions thereof, can be stored in the remote memory/storage device 1352. It will be appreciated that the network connections shown are example and other means of establishing a communications link between the computers can be used.

When used in either a LAN or WAN networking environment, the computer 1302 can access cloud storage systems or other network-based storage systems in addition to, or in place of, external storage devices 1316 as described above, such as but not limited to a network virtual machine providing one or more aspects of storage or processing of information. Generally, a connection between the computer 1302 and a cloud storage system can be established over a LAN 1354 or WAN 1356 e.g., by the adapter 1358 or modem 1360, respectively. Upon connecting the computer 1302 to an associated cloud storage system, the external storage interface 1326 can, with the aid of the adapter 1358 or modem 1360, manage storage provided by the cloud storage system as it would other types of external storage. For instance, the external storage interface 1326 can be configured to provide access to cloud storage sources as if those sources were physically connected to the computer 1302.

The computer 1302 can be operable to communicate with any wireless devices or entities operatively disposed in wireless communication, e.g., a printer, scanner, desktop or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

FIG. 14 is a schematic block diagram of a sample computing environment 1400 with which the disclosed subject matter can interact. The sample computing environment 1400 includes one or more client(s) 1410. The client(s) 1410 can be hardware or software (e.g., threads, processes, computing devices). The sample computing environment 1400 also includes one or more server(s) 1430. The server(s) 1430 can also be hardware or software (e.g., threads, processes, computing devices). The servers 1430 can house threads to perform transformations by employing one or more embodiments as described herein, for example. One possible communication between a client 1410 and a server 1430 can be in the form of a data packet adapted to be transmitted between two or more computer processes. The sample computing environment 1400 includes a communication framework 1450 that can be employed to facilitate communications between the client(s) 1410 and the server(s) 1430. The client(s) 1410 are operably connected to one or more client data store(s) 1420 that can be employed to store information local to the client(s) 1410. Similarly, the server(s) 1430 are operably connected to one or more server data store(s) 1440 that can be employed to store information local to the servers 1430.

Various embodiments may be a system, a method, an apparatus or a computer program product at any possible technical detail level of integration. The computer program product can include a computer readable storage medium (or media) having computer readable program instructions thereon for causing a processor to carry out aspects of various embodiments. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network or a wireless network. The network can comprise copper transmission cables, optical transmission fibers, wireless transmission, routers, firewalls, switches, gateway computers or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of various embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language, the Python programming language, or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform various aspects.

Various aspects are described herein with reference to flowchart illustrations or block diagrams of methods, apparatus (systems), and computer program products according to various embodiments. It will be understood that each block of the flowchart illustrations or block diagrams, and combinations of blocks in the flowchart illustrations or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart or block diagram block or blocks. These computer readable program instructions can also be stored in a computer readable storage medium that can direct a computer, a programmable data processing apparatus, or other devices to function in a particular manner, such that the computer readable storage medium having instructions stored therein comprises an article of manufacture including instructions which implement aspects of the function/act specified in the flowchart or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart or block diagram block or blocks.

The flowcharts and block diagrams in the Figures illustrate the architecture, functionality, and operation of possible implementations of systems, methods, and computer program products according to various embodiments. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer or computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that various aspects can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, or a computer. By way of illustration, both an application running on a server and the server can be a component. One or more components can reside within a process or thread of execution and a component can be localized on one computer or distributed between two or more computers. In another example, respective components can execute from various computer readable media having various data structures stored thereon. The components can communicate via local or remote processes such as in accordance with a signal having one or more data packets (e.g., data from one component interacting with another component in a local system, distributed system, or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. As used herein, the term “and/or” is intended to have the same meaning as “or.” Moreover, articles “a” and “an” as used in the subject specification and annexed drawings should generally be construed to mean “one or more” unless specified otherwise or clear from context to be directed to a singular form. As used herein, the terms “example” or “exemplary” are utilized to mean serving as an example, instance, or illustration. For the avoidance of doubt, the subject matter disclosed herein is not limited by such examples. In addition, any aspect or design described herein as an “example” or “exemplary” is not necessarily to be construed as preferred or advantageous over other aspects or designs, nor is it meant to preclude equivalent exemplary structures and techniques known to those of ordinary skill in the art.

The herein disclosure describes non-limiting examples. For ease of description or explanation, various portions of the herein disclosure utilize the term “each,” “every,” or “all” when discussing various examples. Such usages of the term “each,” “every,” or “all” are non-limiting. In other words, when the herein disclosure provides a description that is applied to “each,” “every,” or “all” of some particular object or component, it should be understood that this is a non-limiting example, and it should be further understood that, in various other examples, it can be the case that such description applies to fewer than “each,” “every,” or “all” of that particular object or component.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system for segmenting medical image data, the system comprising:
a memory that stores computer executable components; and
a processor that executes at least one of the computer executable components that:
collects one or more weak annotations for the medical image data;
applies an interpolation technique to propagate the one or more weak annotations to unannotated regions of the medical image data to generate propagated annotations;
trains a segmentation model using a training dataset comprising the one or more weak annotations, the propagated annotations, and the medical image data; and
iteratively retrains the segmentation model, wherein an iteration of retraining comprises:
generating a set of pseudo labels for a subset of unannotated regions of the medical image data using the segmentation model;
adding the set of pseudo labels to the training dataset; and
updating one or more parameters of the segmentation model using the training dataset,
wherein the set of pseudo labels generated from successive iterations are retained in the training dataset such that a number of annotations associated with the medical image data in the training dataset increases across successive iterations.

2. The system of claim 1, wherein the at least one of the computer executable components further:
collects one or more precise annotations for the medical image data.

3. The system of claim 2, wherein the at least one of the computer executable components further:
trains the segmentation model using the one or more precise annotations with the one or more weak annotations.

4. The system of claim 1, wherein the one or more weak annotations comprise different annotation types associated with the medical image data.

5. The system of claim 1, wherein the one or more weak annotations comprise point-based annotations, and wherein the applying the interpolation technique to propagate the one or more weak annotations to unannotated regions of the medical image data comprises:
assigning labels to regions of the medical image data within a predefined radius of one or more point-based annotations based on the one or more weak annotations or the set of pseudo labels.

6. The system of claim 1, wherein the at least one of the computer executable components further:
selects a size or thickness of the one or more weak annotations.

7. The system of claim 1, wherein the one or more weak annotations comprise slice-based annotations, and wherein the at least one of the computer executable components further:
assigns a background label to negative slices of the medical image data.

8. The system of claim 1, wherein the at least one of the computer executable components further:
iteratively trains the segmentation model on the set of pseudo labels until a segmentation accuracy of the segmentation model meets a predetermined threshold.

9. The system of claim 1, wherein the at least one of the computer executable components further:
iteratively trains a second segmentation model on the medical image data, the one or more weak annotations, and the set of pseudo labels.

10. A computer-implemented method, comprising:
collecting, by a system operatively coupled to a processor, one or more weak annotations for medical image data;
applying, by the system, an interpolation technique to propagate the one or more weak annotations to unannotated regions of the medical image data to generate propagated annotations;
training, by the system, a segmentation model using a training dataset comprising the one or more weak annotations, the propagated annotations, and the medical image data; and
iteratively retraining, by the system, the segmentation model, wherein an iteration of retraining comprises:
generating a set of pseudo labels for a subset of unannotated regions of the medical image data using the segmentation model;
adding the set of pseudo labels to the training dataset; and
updating one or more parameters of the segmentation model using the training dataset,
wherein the set of pseudo labels generated from successive iterations are retained in the training dataset such that a number of annotations associated with the medical image data in the training dataset increases across successive iterations.

11. The computer-implemented method of claim 10, further comprising:
collecting, by the system, one or more precise annotations for the medical image data.

12. The computer-implemented method of claim 11, further comprising:
training, by the system, the segmentation model using the one or more precise annotations with the one or more weak annotations.

13. The computer-implemented method of claim 10, wherein the one or more weak annotations comprise different annotation types associated with the medical image data.

14. The computer-implemented method of claim 10, wherein the one or more weak annotations comprise point-based annotations, and wherein the applying the interpolation technique to propagate the one or more weak annotations to unannotated regions of the medical image data comprises:
assigning labels to regions of the medical image data within a predefined radius of one or more point-based annotations based on the one or more weak annotations or the set of pseudo labels.

15. The computer-implemented method of claim 10, further comprising:
selecting, by the system, a size or thickness of the one or more weak annotations.

16. The computer-implemented method of claim 10, wherein the one or more weak annotations comprise slice-based annotations, and further comprising:
assigning, by the system, a background label to negative slices of the medical image data.

17. The computer-implemented method of claim 10, further comprising:
iteratively training, by the system, the segmentation model on the set of pseudo labels until a segmentation accuracy of the segmentation model meets a predetermined threshold.

18. The computer-implemented method of claim 10, further comprising:
iteratively training, by the system, a second segmentation model on the medical image data, the one or more weak annotations, and the set of pseudo labels.

19. A computer program product for weakly supervised segmentation of medical image data, the computer program product comprising a non-transitory computer-readable memory having program instructions embodied therewith, the program instructions executable by a processor to cause the processor to:
collect, by the processor, one or more weak annotations for the medical image data;
apply, by the processor, an interpolation technique to propagate the one or more weak annotations to unannotated regions of the medical image data to generate propagated annotations;
train, by the processor, a segmentation model using a training dataset comprising the one or more weak annotations, the propagated annotations, and the medical image data; and
iteratively retrain, by the processor, the segmentation model, wherein an iteration of retraining comprises:
generating a set of pseudo labels for a subset of unannotated regions of the medical image data using the segmentation model;
adding the set of pseudo labels to the training dataset; and
updating one or more parameters of the segmentation model using the training dataset,
wherein the set of pseudo labels generated from successive iterations are retained in the training dataset such that a number of annotations associated with the medical image data in the training dataset increases across successive iterations.

20. The computer program product of claim 19, wherein the program instructions are further executable to cause the processor to:
iteratively train, by the processor, the segmentation model on the set of pseudo labels until a segmentation accuracy of the segmentation model meets a predetermined threshold.
